SEQUENCE LISTING (184 sequences)

SEQ ID NO: 1 - DNA, 1161 bp, synthetic construct
  source 1..1161: mol_type = other DNA
gccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatgggataataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcgatagcacttggtttgattccaccggtgggagcggaagtggcggttccggaccttcaaagagatctttcattgaagacctgcttttcaacaaggtctaatga

SEQ ID NO: 2 - protein, 383 aa, synthetic construct
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGSGPSKRSFIEDLLFNKV

SEQ ID NO: 3 - DNA, 1236 bp, synthetic construct
  source 1..1236: mol_type = other DNA
gccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatgggataataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcgatagcacttggtttgattccaccggtgggagcggaagtggcggttccggatcattcattgaagaccttctctttaacaaggtgaccctcgccgatgcaggtttcattaagcaatatggtgattgcctgggagacatcgcggctcgtgatcttatttgtgcgcagaaattttaatga

SEQ ID NO: 4 - protein, 408 aa, synthetic construct
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGSGSFIEDLLFNKVTLADAGFIKQYGDCLGDIAARDLICAQKF

SEQ ID NO: 5 - DNA, 1161 bp, synthetic construct
  source 1..1161: mol_type = other DNA
gccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatgggataataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcgatagcacttggtttgattccaccggtgggagcggaagtggcggttccggagtcgcaggcagatctgcgattgaagacatccttttcagcaagctttaatga

SEQ ID NO: 6 - protein, 383 aa, synthetic construct
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGSGVAGRSAIEDILFSKL

SEQ ID NO: 7 - DNA, 1158 bp, synthetic construct
  source 1..1158: mol_type = other DNA
gccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatgggataataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcgatagcacttggtttgattccaccggtgggagcggaagtggcggttccggatcagcaagatctgcaattgaagacctgcttttcgataaggtctaatga

SEQ ID NO: 8 - protein, 382 aa, synthetic construct
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGSGSARSAIEDLLFDKV

SEQ ID NO: 9 - DNA, 1161 bp, synthetic construct
  source 1..1161: mol_type = other DNA
gccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatgggataataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcgatagcacttggtttgattccaccggtgggagcggaagtggcggttccggaatcgcaggcagatctgcgctggaagacctgcttttcagcaaggtctaatga

SEQ ID NO: 10 - protein, 383 aa, synthetic construct
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGSGIAGRSALEDLLFSKV

SEQ ID NO: 11 - DNA, 1206 bp, synthetic construct
  source 1..1206: mol_type = other DNA
gccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatgggataataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcgatagcacttggtttgattccaccggtgggagcggaagtggcggttccggaccttcaaagagatctttcattgaagacctgcttttcaacaaggtcccctctaaacggagctttatcgaggatctcctctttaataaagtgtaatga

SEQ ID NO: 12 - protein, 398 aa, synthetic construct
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGSGPSKRSFIEDLLFNKVPSKRSFIEDLLFNKV

SEQ ID NO: 13 - DNA, 1283 bp, synthetic construct
  source 1..1283: mol_type = other DNA
gccaccatggactggacctggattctgttcctggtggccgccgccacaagggtgcacagcccagtaagcgctccttcatcgaagatctcctcttcaacaaagttgcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagctgataa

SEQ ID NO: 14 - protein, 424 aa, synthetic construct
MDWTWILFLVAAATRVHSPSKRSFIEDLLFNKVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 15 - DNA, 1283 bp, synthetic construct
  source 1..1283: mol_type = other DNA
gccaccatggactggacctggattctgttcctggtggccgccgccacaagggtgcacagcctaccaagagatctttcattgaagacctgcttttcaacaaggtcgcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagctgataa

SEQ ID NO: 16 - protein, 424 aa, synthetic construct
MDWTWILFLVAAATRVHSPTKRSFIEDLLFNKVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 17 - DNA, 1280 bp, synthetic construct
  source 1..1280: mol_type = other DNA
gccaccatggactggacctggattctgttcctggtggccgccgccacaagggtgcacagtcagcaagatctgcaattgaagacctgcttttcgataaggtcgcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagctgataa

SEQ ID NO: 18 - protein, 423 aa, synthetic construct
MDWTWILFLVAAATRVHSSARSAIEDLLFDKVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 19 - DNA, 1283 bp, synthetic construct
  source 1..1283: mol_type = other DNA
gccaccatggactggacctggattctgttcctggtggccgccgccacaagggtgcacaggcgtcaagtagatctgcaattgaagacctgcttttcgataaggtcgcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagctgataa

SEQ ID NO: 20 - protein, 424 aa, synthetic construct
MDWTWILFLVAAATRVHSASSRSAIEDLLFDKVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 21 - DNA, 1283 bp, synthetic construct
  source 1..1283: mol_type = other DNA
gccaccatggactggacctggattctgttcctggtggccgccgccacaagggtgcacagagctcatctagatctcttctggaagacctgcttttcaacaaggtcgcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagctgataa

SEQ ID NO: 22 - protein, 424 aa, synthetic construct
MDWTWILFLVAAATRVHSSSSRSLLEDLLFNKVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 23 - DNA, 1283 bp, synthetic construct
  source 1..1283: mol_type = other DNA
gccaccatggactggacctggattctgttcctggtggccgccgccacaagggtgcacaggtcgcaggcagatctgcgattgaagacatccttttcagcaagcttgcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagctgataa

SEQ ID NO: 24 - protein, 424 aa, synthetic construct
MDWTWILFLVAAATRVHSVAGRSAIEDILFSKLAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 25 - DNA, 1273 bp, synthetic construct
  source 1..1273: mol_type = other DNA
gccaccatggactggacctggattctgttcctggtggccgccgccacaagggtgcacaggatctgcgctggaagacctgcttttcagcaaggtcgcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagctgataa

SEQ ID NO: 26 - protein, 424 aa, synthetic construct
MDWTWILFLVAAATRVHSIAGRSALEDLLFSKVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 27 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacctagcaagcggtcctttattgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 28 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 29 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagcagggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacctagcaagcggtcctttattgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggagccgctctgcagatccccttcgcaatgcagatggcatacagattcaacggaattggagtcactcagaatgttctgtacgagaatcaaaagttgattgccaaccagtttaactccgcaattggtaagatacaagacagcctctcttctactgcatccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 30 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNEQGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 31 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagcagggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactcagacttccaatcaggtagcggtcctttaccaggacgttaattgtgccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttgcccagattctccccgacccttccaaacctagcaagcggtcctttattgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 32 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNEQGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTQTSNQVAVLYQDVNCAEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFAQILPDPSKPSKRSFIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 33 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaagtggccggtcggtccgcaattgaggatatacttttctccaaactcaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 34 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKVAGRSAIEDILFSKLTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 35 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaaagtagctcccggtccctgctcgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 36 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKSSSRSLLEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 37 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacctagcgcccggtccgccattgaggatctccttttcgacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 38 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSARSAIEDLLFDKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 39 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaaatagccggtcggtccgccctggaggatctccttttctccaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 40 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKIAGRSALEDLLFSKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 41 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaagccagctcccggtccgccattgaggatctccttttcgacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 42 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKASSRSAIEDLLFDKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 43 - DNA, 3765 bp, synthetic construct
  source 1..3765: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacggtcctttattgaggatctccttttctggaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 44 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFWKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 45 - DNA, 3765 bp, synthetic construct
  source 1..3765: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacggtcctttattgaggatctccttttcaacaaaacaaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 46 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKTTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 47 - DNA, 3774 bp, synthetic construct
  source 1..3774: mol_type = other DNA
gccaccatggattggacgtggatacttttccttgttgctgctgctacccgcgtacacagcatgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaaattgtgtttaacttcctcagccctaagcgggaggattccaaacttaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 48 - protein, 1254 aa, synthetic construct
MDWTWILFLVAAATRVHSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKIVFNFLSPKREDSKLTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 49 - DNA, 54 bp, synthetic construct
  source 1..54: mol_type = other DNA
atggattggacgtggatacttttccttgttgctgctgctacccgcgtacacagc

SEQ ID NO: 50 - protein, 18 aa, synthetic construct
MDWTWILFLVAAATRVHS

SEQ ID NO: 51 - DNA, 27 bp, synthetic construct
  source 1..27: mol_type = other DNA
ggtgggagcggaagtggcggttccgga

SEQ ID NO: 52 - protein, 9 aa, synthetic construct
GGSGSGGSG

SEQ ID NO: 53 - DNA, 1024 bp, synthetic construct
  source 1..1024: mol_type = other DNA
catgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatgggataataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcgatagcacttggtttgattccacc

SEQ ID NO: 54 - protein, 341 aa, synthetic construct
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDST

SEQ ID NO: 55 - DNA, 45 bp, synthetic construct
  source 1..45: mol_type = other DNA
ccttcaaagagatctttcattgaagacctgcttttcaacaaggtc

SEQ ID NO: 56 - protein, 15 aa, synthetic construct
PSKRSFIEDLLFNKV

SEQ ID NO: 57 - DNA, 120 bp, synthetic construct
  source 1..120: mol_type = other DNA
tcattcattgaagaccttctctttaacaaggtgaccctcgccgatgcaggtttcattaagcaatatggtgattgcctgggagacatcgcggctcgtgatcttatttgtgcgcagaaattt

SEQ ID NO: 58 - protein, 40 aa, synthetic construct
SFIEDLLFNKVTLADAGFIKQYGDCLGDIAARDLICAQKF

SEQ ID NO: 59 - DNA, 45 bp, synthetic construct
  source 1..45: mol_type = other DNA
gtcgcaggcagatctgcgattgaagacatccttttcagcaagctt

SEQ ID NO: 60 - protein, 15 aa, synthetic construct
VAGRSAIEDILFSKL

SEQ ID NO: 61 - DNA, 42 bp, synthetic construct
  source 1..42: mol_type = other DNA
tcagcaagatctgcaattgaagacctgcttttcgataaggtc

SEQ ID NO: 62 - protein, 14 aa, synthetic construct
SARSAIEDLLFDKV

SEQ ID NO: 63 - DNA, 45 bp, synthetic construct
  source 1..45: mol_type = other DNA
atcgcaggcagatctgcgctggaagacctgcttttcagcaaggtc

SEQ ID NO: 64 - protein, 15 aa, synthetic construct
IAGRSALEDLLFSKV

SEQ ID NO: 65 - DNA, 90 bp, synthetic construct
  source 1..90: mol_type = other DNA
ccttcaaagagatctttcattgaagacctgcttttcaacaaggtcccctctaaacggagctttatcgaggatctcctctttaataaagtg

SEQ ID NO: 66 - protein, 30 aa, synthetic construct
PSKRSFIEDLLFNKVPSKRSFIEDLLFNKV

SEQ ID NO: 67 - DNA, 1173 bp, synthetic construct
  source 1..1173: mol_type = other DNA
gcccccctgcaccttgggaagtgtaacatcgcaggctggattctgggcaacccagagtgtgagagcctttcaacagcctcttcctggagctacattgttgagactccaagtagtgacaacgggacctgctttccaggggacttcattgattatgaggagctccgggaacagttgtcctctgtatcttcatttgaaaggtttgagatatttccgaagacgtcgagctggccaaaccacgattcaaataaaggtgtcacagctgcctgccctcatgccggcgccaaaagtttctacaagaacctgatctggctggtcaagaaaggcaacagttatcccaaactatcaaagtcttacattaatgacaagggaaaagaagtgttagtcctgtggggcatccaccacccaagcacctctgcggatcaacagtcactgtaccagaatgcagacacctatgtgtttgtggggtcctcgcgctactccaaaaagttcaagcctgaaatagcaatacgtccgaaagtgagagaccaggagggaagaatgaactactactggacactggtggagcctggagataaaatcaccttcgaagctactgggaatctagtcgttccccgatatgcttttgccatggagcggaatgcttctggagagagccaagtgaggcagcagttctccaaggacattgagaagcttctgaatgagcaagtgaacaaggagatgcagagctccaatctgtacatgtccatgagcagttggtgctacactcacagcttggatggtgctggcctcttcctttttgatcatgctgctgaggaatatgaacatgccaagaagctgattatcttccttaacgagaacaatgtacctgtgcagctcactagcatctctgcgcctgagcacaagtttgaaggtttaacgcagatcttccagaaggcatatgaacatgagcagcacatctctgagtcaatcaacaacattgtggaccacgccatcaagagtaaagaccatgcaacatttaacttcctgcagtggtatgtcgcagaacaacatgaagaagaggttttgtttaaagatattttagataaaattgaactgattggaaatgaaaatcatggcctctatctggctgaccagtacgtcaaaggaatagcaaaaagtaggaagtcaggtagc

SEQ ID NO: 68 - protein, 391 aa, synthetic construct
APLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 69 - DNA, 45 bp, synthetic construct
  source 1..45: mol_type = other DNA
cccagtaagcgctccttcatcgaagatctcctcttcaacaaagtt

SEQ ID NO: 70 - protein, 15 aa, synthetic construct
PSKRSFIEDLLFNKV

SEQ ID NO: 71 - DNA, 45 bp, synthetic construct
  source 1..45: mol_type = other DNA
cctaccaagagatctttcattgaagacctgcttttcaacaaggtc

SEQ ID NO: 72 - protein, 15 aa, synthetic construct
PTKRSFIEDLLFNKV

SEQ ID NO: 73 - DNA, 45 bp, synthetic construct
  source 1..45: mol_type = other DNA
gcgtcaagtagatctgcaattgaagacctgcttttcgataaggtc

SEQ ID NO: 74 - protein, 15 aa, synthetic construct
ASSRSAIEDLLFDKV

SEQ ID NO: 75 - DNA, 45 bp, synthetic construct
  source 1..45: mol_type = other DNA
agctcatctagatctcttctggaagacctgcttttcaacaaggtc

SEQ ID NO: 76 - protein, 15 aa, synthetic construct
SSSRSLLEDLLFNKV

SEQ ID NO: 77 - DNA, 35 bp, synthetic construct
  source 1..35: mol_type = other DNA
gatctgcgctggaagacctgcttttcagcaaggtc

SEQ ID NO: 78 - protein, 15 aa, synthetic construct
IAGRSALEDLLFSKV

SEQ ID NO: 79 - DNA, 3708 bp, synthetic construct
  source 1..3708: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacctagcaagcggtcctttattgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcgga

SEQ ID NO: 80 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 81 - DNA, 3708 bp, synthetic construct
  source 1..3708: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagcagggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacctagcaagcggtcctttattgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggagccgctctgcagatccccttcgcaatgcagatggcatacagattcaacggaattggagtcactcagaatgttctgtacgagaatcaaaagttgattgccaaccagtttaactccgcaattggtaagatacaagacagcctctcttctactgcatccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcgga

SEQ ID NO: 82 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNEQGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 83 - DNA, 3708 bp, synthetic construct
  source 1..3708: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagcagggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactcagacttccaatcaggtagcggtcctttaccaggacgttaattgtgccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttgcccagattctccccgacccttccaaacctagcaagcggtcctttattgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcgga

SEQ ID NO: 84 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNEQGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTQTSNQVAVLYQDVNCAEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFAQILPDPSKPSKRSFIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 85 - DNA, 45 bp, synthetic construct
  source 1..45: mol_type = other DNA
gtggccggtcggtccgcaattgaggatatacttttctccaaactc

SEQ ID NO: 86 - protein, 15 aa, synthetic construct
VAGRSAIEDILFSKL

SEQ ID NO: 87 - DNA, 3714 bp, synthetic construct
  source 1..3714: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaagtggccggtcggtccgcaattgaggatatacttttctccaaactcaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 88 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKVAGRSAIEDILFSKLTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 89 - DNA, 45 bp, synthetic construct
  source 1..45: mol_type = other DNA
agtagctcccggtccctgctcgaggatctccttttcaacaaagtg

SEQ ID NO: 90 - protein, 15 aa, synthetic construct
SSSRSLLEDLLFNKV

SEQ ID NO: 91 - DNA, 3714 bp, synthetic construct
  source 1..3714: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaaagtagctcccggtccctgctcgaggatctccttttcaacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 92 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKSSSRSLLEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 93 - DNA, 45 bp, synthetic construct
  source 1..45: mol_type = other DNA
cctagcgcccggtccgccattgaggatctccttttcgacaaagtg

SEQ ID NO: 94 - protein, 15 aa, synthetic construct
PSARSAIEDLLFDKV

SEQ ID NO: 95 - DNA, 3714 bp, synthetic construct
  source 1..3714: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacctagcgcccggtccgccattgaggatctccttttcgacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 96 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSARSAIEDLLFDKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 97 - DNA, 45 bp, synthetic construct
  source 1..45: mol_type = other DNA
atagccggtcggtccgccctggaggatctccttttctccaaagtg

SEQ ID NO: 98 - protein, 15 aa, synthetic construct
IAGRSALEDLLFSKV

SEQ ID NO: 99 - DNA, 3708 bp, synthetic construct
  source 1..3708: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaaatagccggtcggtccgccctggaggatctccttttctccaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcgga

SEQ ID NO: 100 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKIAGRSALEDLLFSKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 101 - DNA, 45 bp, synthetic construct
  source 1..45: mol_type = other DNA
gccagctcccggtccgccattgaggatctccttttcgacaaagtg

SEQ ID NO: 102 - protein, 15 aa, synthetic construct
ASSRSAIEDLLFDKV

SEQ ID NO: 103 - DNA, 3714 bp, synthetic construct
  source 1..3714: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaagccagctcccggtccgccattgaggatctccttttcgacaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 104 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKASSRSAIEDLLFDKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 105 - DNA, 36 bp, synthetic construct
  source 1..36: mol_type = other DNA
cggtcctttattgaggatctccttttctggaaagtg

SEQ ID NO: 106 - protein, 15 aa, synthetic construct
PSKRSFIEDLLFWKV

SEQ ID NO: 107 - DNA, 3705 bp, synthetic construct
  source 1..3705: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacggtcctttattgaggatctccttttctggaaagtgaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 108 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFWKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 109 - DNA, 36 bp, synthetic construct
  source 1..36: mol_type = other DNA
cggtcctttattgaggatctccttttcaacaaaaca

SEQ ID NO: 110 - protein, 15 aa, synthetic construct
PSKRSFIEDLLFNKT

SEQ ID NO: 111 - DNA, 3705 bp, synthetic construct
  source 1..3705: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaacggtcctttattgaggatctccttttcaacaaaacaaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 112 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKTTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 113 - DNA, 45 bp, synthetic construct
  source 1..45: mol_type = other DNA
attgtgtttaacttcctcagccctaagcgggaggattccaaactt

SEQ ID NO: 114 - protein, 15 aa, synthetic construct
IVFNFLSPKREDSKL

SEQ ID NO: 115 - DNA, 3714 bp, synthetic construct
  source 1..3714: mol_type = other DNA
atgttcgtcttcctggtgttgctccccctggtttccagccagtgcgtgaacctcacaactcgtacccagctcccacccgcttacaccaactcctttaccaggggcgtgtattacccagataaggtgtttcgtagcagcgtgctgcacagtacccaggacctgtttctccctttcttcagcaatgtcacatggtttcacgccatccacgtttctggtactaatggtaccaagcgtttcgacaaccccgtgctcccgtttaacgatggtgtctacttcgcttccaccgaaaagtccaacattatccgtggctggatctttggcaccaccctggatagcaaaacacagtcactgctgattgtgaataacgccaccaacgtcgtgataaaagtgtgcgaattccagttttgcaatgatcctttcctgggagtttattatcacaagaacaacaaaagttggatggaatccgagttccgggtttattcctctgcaaataactgcaccttcgagtacgtctctcagccttttctcatggacctcgaagggaaacaaggaaacttcaagaatctgagggaatttgttttcaagaacattgacgggtatttcaagatctacagcaagcatacccccattaacctcgtgagggacctgccacaaggcttctccgccttggaaccactcgtcgacctgcctataggcattaacatcacccgatttcagacgctcctggcattgcaccgaagctatctgacaccaggtgattcatcctctggctggacggccggtgcggcagcttattacgtgggatatctgcagcctaggacattcctgctcaagtataatgagaacggcactatcactgatgcagtggactgtgcccttgacccgctcagtgagaccaagtgcaccctcaagtccttcactgttgaaaagggtatctatcaaaccagcaattttagagtccagcctactgagagcattgtcagatttcctaatatcacaaatctctgtccttttggtgaggtctttaatgcgacccgcttcgccagtgtgtatgcctggaatcgtaagagaattagcaactgtgtagcagactattcagtcttgtacaactccgccagcttttctacatttaagtgttacggggtgtccccgaccaagttgaacgatttgtgcttcacaaacgtttacgccgatagctttgttattaggggagatgaagttcgccagattgcccctggccaaaccgggaaaattgcggactacaactataagttgcccgacgacttcactggttgtgtaatcgcctggaactccaacaatctggacagtaaggtaggtggcaattataactatttgtatcgccttttccggaagtccaatctgaaacccttcgaacgggacatttcaactgaaatctaccaggccggaagtacgccatgtaacggcgttgaggggttcaattgttacttcccactgcaaagctacggtttccagccaactaatggagttggctatcagccttaccgggtggtggttctttccttcgagcttttgcatgctccagccacggtctgcggaccaaagaagtcaacaaacttggtgaagaacaaatgcgtaaacttcaactttaacggtctcactggcacaggagtgcttacggagtccaataagaagttccttccttttcagcagtttggtcgagacatcgctgatacaacggatgctgtaagagacccccagactttggagattctggacattaccccatgctcatttggcggcgtgtcagtgatcaccccaggtactaacacttccaatcaggtagcggtcctttaccaggacgttaattgtaccgaagtacccgtagcgatccacgcagaccagctgactcccacttggcgggtttacagtacgggttcaaacgttttccagacccgtgctggatgccttatcggtgcggagcatgtaaacaactcatacgaatgtgacatccctatcggggcaggaatatgcgcatcttaccaaacacagacaaattctccaggctcagcatcttccgtggcgtctcagagtataatcgcatataccatgagtcttggtgccgagaattcagtcgcgtattctaacaattctatcgctatcccaacgaacttcacaatatccgtcacaaccgagattctccctgtttctatgactaagactagtgtggattgtacgatgtatatctgtggggatagcacagaatgcagtaacctgctgcttcagtacggctcattttgtacccaactcaatagagccctcaccggtatagccgtggaacaggataagaatactcaagaggtgtttgcccaggtgaagcaaatttataaaactccacctattaaggacttcggtggatttaattttagtcagattctccccgacccttccaaaattgtgtttaacttcctcagccctaagcgggaggattccaaacttaccctggccgacgccgggttcattaaacagtacggagactgcctcggtgacatagccgcacgcgatcttatctgtgcacagaaatttaacggtcttaccgtccttccgccactgttgacagatgaaatgatcgcccaatacacctcagcattgctggctggtaccattacttctggatggacatttggtgcaggacccgctctgcagatccccttccctatgcagatggcatacagattcaacggaattggagtcactcagaatgttctgtacgagaatcaaaagttgattgccaaccagtttaactccgcaattggtaagatacaagacagcctctcttctactccctccgcgttgggaaagctgcaggatgtggtgaatcagaacgcacaggccctgaatactctggtgaagcagttgagctcaaactttggggccatctcttccgtcctcaatgacatactgagcagactcgatcccccagaggctgaggtgcagattgatcgcttgatcacggggagactccagtctctccagacatatgtaactcagcagctgattagagcggcggagatcagagcatccgccaaccttgccgctaccaaaatgtctgaatgcgtgctgggccagtccaagcgtgtggatttctgtgggaagggatatcatctgatgtcatttccccaatcagctcctcacggcgttgttttcctgcatgttacctacgtgccagctcaggagaagaatttcactaccgcgcctgccatttgccatgacggcaaggcgcatttccccagggaaggagtctttgttagtaatggtacacactggttcgtcacccagcgtaatttctacgagccacagattattacgacagacaacactttcgtatccggtaattgcgacgtagtcattggcattgtaaataataccgtatacgaccctctccagcccgaacttgacagttttaaggaagagttggataaatacttcaagaaccatacttctccagatgtcgacctgggtgatatttctggaatcaatgcctctgttgttaacattcagaaagaaatagatcggctcaatgaggtcgccaagaatttgaacgagtcactgattgacctgcaggagctcggaaagtatgaacagggatatataccagaggcccccagagatggccaggcctatgttcgcaaggacggggagtgggtcctgctgtccactttcctcggataataa

SEQ ID NO: 116 - protein, 1236 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKIVFNFLSPKREDSKLTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGYIPEAPRDGQAYVRKDGEWVLLSTFLG

SEQ ID NO: 117 - DNA, 354 bp, synthetic construct
  source 1..354: mol_type = other DNA
gaagtgaaactggtggagtctgggggaggcttagtgaagcctggagggtctctgaaactctcctgtgtagcctctggattcactttcagtaacgacgccatgtcttgggttcgccagactccagagaagaggctggagtgggtcgcatccatcagtagtggtggtaggaagtactatccagacaatgtgaagggccgattcaccatctccagagataatgccaggaacatcctgtacctgcaaatgagcagtctgaggtctgaggacacggccatgtattattgtgcaagagaggggatctacgtctgggctatggactactggggtcagggaacctcagtcaccgtctcctca

SEQ ID NO: 118 - protein, 118 aa, synthetic construct
EVKLVESGGGLVKPGGSLKLSCVASGFTFSNDAMSWVRQTPEKRLEWVASISSGGRKYYPDNVKGRFTISRDNARNILYLQMSSLRSEDTAMYYCAREGIYVWAMDYWGQGTSVTVSS

SEQ ID NO: 119 - DNA, 336 bp, synthetic construct
  source 1..336: mol_type = other DNA
gatattgtgatgacgcaggctgcattctccaatccagtcactcttggaacatcagcttccatctcctgcaggtctagtgagagtctcctccatagtaatggcatcacttatttgtattggtatctgcagaagccaggccagtctcctcagctcctgatttatcggatgtccaaccttgcctcaggagtcccagacaggttcagtagcagtgggtcaggaactgatttcacactgagaatcagcagagtggaggctgaggatgtgggtgtttattactgtgctcaaaatctagaacttcctccgacgttcggtggaggcaccaaactggaaatcaaa

SEQ ID NO: 120 - protein, 112 aa, synthetic construct
DIVMTQAAFSNPVTLGTSASISCRSSESLLHSNGITYLYWYLQKPGQSPQLLIYRMSNLASGVPDRFSSSGSGTDFTLRISRVEAEDVGVYYCAQNLELPPTFGGGTKLEIK

SEQ ID NO: 121 - DNA, 354 bp, synthetic construct
  source 1..354: mol_type = other DNA
gaagtgaagctggtggagtctgggggaggcttagtgaagcctggagggtccctgaaactctcctgtgcagcctctggaatcactttcagtagttctgccatgtcttgggttcgccagactccagagaagaggctggagtgggtcgcatccattagtagcggtggtcatacctactatctagacagtatgaagggccgattcaccatctccagagataatgccaggaacatcctgtacctacaaatgggaagtctgaggtctgaggacacggccgtgtattattgtgcaagagaagggatctacgtctgggctatggactactggggtcaaggaacctcagtcaccgtctcctca

SEQ ID NO: 122 - protein, 118 aa, synthetic construct
EVKLVESGGGLVKPGGSLKLSCAASGITFSSSAMSWVRQTPEKRLEWVASISSGGHTYYLDSMKGRFTISRDNARNILYLQMGSLRSEDTAVYYCAREGIYVWAMDYWGQGTSVTVSS

SEQ ID NO: 123 - DNA, 336 bp, synthetic construct
  source 1..336: mol_type = other DNA
gatattgtgatgacgcaggctgcattctccaatccagtcactcttggaacatcagcttccatctcctgcaggtctagtaagagtctcctacaaagtaatggcatcacttatttgtattggtatctgcagaagccaggccagtcccctcagctcctgatttatcggatgtccaaccttgcctcaggagtcccagacaggttcagtggcagtgggtcaggaactgatttcacactgagaatcagcagagtggaggctgaggatgtgggtatttactactgtgctcaaaatctagaacttcctccgacgttcggtggaggcaccaagctggaaatcaaa

SEQ ID NO: 124 - protein, 112 aa, synthetic construct
DIVMTQAAFSNPVTLGTSASISCRSSKSLLQSNGITYLYWYLQKPGQSPQLLIYRMSNLASGVPDRFSGSGSGTDFTLRISRVEAEDVGIYYCAQNLELPPTFGGGTKLEIK

SEQ ID NO: 125 - DNA, 354 bp, synthetic construct
  source 1..354: mol_type = other DNA
gaagtgaaactggtggagtctgggggaggcttagtgaagcctggagggtctctgaaactctcctgtctagtctctggaatcactttcagtaacgacgccatgtcttgggttcgccagactccagagaagaggctggagtgggtcgcatccattagtagtggtggtaggaattactatccagatagtgtgaagggccgattcaccatctccagagataatgccaggaacatcctgtacctgcaaatgagcagtctgaggtctgaggacacggccatgtatttttgtgcaagagaggggatctccgtctgggctatggactactggggtcagggaacctcagtcactgtctcctca

SEQ ID NO: 126 - protein, 118 aa, synthetic construct
EVKLVESGGGLVKPGGSLKLSCLVSGITFSNDAMSWVRQTPEKRLEWVASISSGGRNYYPDSVKGRFTISRDNARNILYLQMSSLRSEDTAMYFCAREGISVWAMDYWGQGTSVTVSS

SEQ ID NO: 127 - DNA, 336 bp, synthetic construct
  source 1..336: mol_type = other DNA
gatattgtgatgacgcaggctgcattctccaatccagtcactcttggaacatcagcttccatctcctgcaggtctagtaagagtctcctccatagtaatggcatcacttatttgtattggtatctgcagaggccaggccagtctcctcagctcctgatttatcggatgtccaaccttgcctcaggagtcccagacaggttcagtagcagtgggtcaggaactgatttcacactgagaatcagcagagtggaggctgaggatgtgggtgtttattactgtgctcaaaatctagaacttcctccgacgttcggtggaggcaccaagctggaaatcaaa

SEQ ID NO: 128 - protein, 112 aa, synthetic construct
DIVMTQAAFSNPVTLGTSASISCRSSKSLLHSNGITYLYWYLQRPGQSPQLLIYRMSNLASGVPDRFSSSGSGTDFTLRISRVEAEDVGVYYCAQNLELPPTFGGGTKLEIK

SEQ ID NO: 129 - DNA, 360 bp, synthetic construct
  source 1..360: mol_type = other DNA
caggtccagctgcagcagtctgggcctgaactagtgaggcctggggaatcagtgaagatttcctgcaagggttccggctacacattcactgattatgctatgcactgggtgaagcagagtcatgctaagagtctagagtggattggaattattagtttttactatgataatacaaagtacaaccagaactttaagggcaaggccacaatgactgtagacaaatcctccagcacagcctatatggaactcgccagattgacatctgaggattctgccatctatttctgtgcaagtcaggggattcattactacggctcgtttgcttactggggccaagggactctggtcactgtctctgca

SEQ ID NO: 130 - protein, 120 aa, synthetic construct
QVQLQQSGPELVRPGESVKISCKGSGYTFTDYAMHWVKQSHAKSLEWIGIISFYYDNTKYNQNFKGKATMTVDKSSSTAYMELARLTSEDSAIYFCASQGIHYYGSFAYWGQGTLVTVSA

SEQ ID NO: 131 - DNA, 321 bp, synthetic construct
  source 1..321: mol_type = other DNA
gacattgtgatgacccagtctcaaaaattcatgtccacatcagtaggagacagggtcagcgtctcctgcaaggccagtcagaatgtgggtactaatttaggctggtatcaacagaaaccagggcaatctcctaaagcactgatttattcggcatcctaccggtacagtggagtccctgatcgcttcacaggcagtggatctgggacagatttcactctcaccatcagcaatgtgcagtctgaagacttggcagagtatttctgtcagcaatataacagctctcctctcacgttcggtgctgggaccaagctggagctgaaa

SEQ ID NO: 132 - protein, 107 aa, synthetic construct
DIVMTQSQKFMSTSVGDRVSVSCKASQNVGTNLGWYQQKPGQSPKALIYSASYRYSGVPDRFTGSGSGTDFTLTISNVQSEDLAEYFCQQYNSSPLTFGAGTKLELK

SEQ ID NO: 133 - protein, 480 aa, synthetic construct
MDWTWILFLVAAATRVHSQVQLQQSGPELVRPGESVKISCKGSGYTFTDYAMHWVKQSHAKSLEWIGIISFYYDNTKYNQNFKGKATMTVDKSSSTAYMELARLTSEDSAIYFCASQGIHYYGSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGKGGEQKLISEEDL

SEQ ID NO: 134 - protein, 482 aa, synthetic construct
MDWTWILFLVAAATRVHSEVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKGGGKPIPNPLLGLDST

SEQ ID NO: 135 - protein, 483 aa, synthetic construct
MDWTWILFLVAAATRVHSEVQLQQSGPVLVKPGASVRMSCKASGYTITDYYLNWVKQSHGKSLEWLGVLNPYSGGSLYSQTFKGKATLTVDRSSSTAYLELNSLTSEDSAVYYCARQLGRGNGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKGGGKPIPNPLLGLDST

SEQ ID NO: 136 - protein, 486 aa, synthetic construct
MDWTWILFLVAAATRVHSQVQLQQSGPGLVKPSQTLSLTCAISGDNVFSNSAAWNWIRQSPSRGLEWLGRTYYRSKWNNDYAVSVKGRITINPDTYKNQFSLQLNPVTPEDTAVYFCARVPMNRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKGGGKPIPNPLLGLDST

SEQ ID NO: 137 - protein, 493 aa, synthetic construct
MDWTWILFLVAAATRVHSQVQLVESGGGVVQPGRSLRLSCAASGLTFSSYVMHWVRQAPGKGLDWVGVIWYDGSKKYYADSVKGRFTISRDNSKNTLYLQMNSLRAEDTAVYYCARDPRDYYDFWSGYDYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKGGGKPIPNPLLGLDST

SEQ ID NO: 138 - protein, 485 aa, synthetic construct
MDWTWILFLVAAATRVHSQVQLVQSGAEVKKPGASVKVSCKVSGYTLIELSMHWVRQAPGKGLEWMGGFDPEDVETIYAQQFQGRVTMTEDTSTDTAYMELSSLRSEDTAVYYCATGWAYKSTWYFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKGGGKPIPNPLLGLDST

SEQ ID NO: 139 - protein, 488 aa, synthetic construct
MDWTWILFLVAAATRVHSQVQLVQSGAEVKKPGSSVKVSCKASGGTFSSYAISWVRQAPGQGLEWMGGNIPIFGTANYAQKFQGRVTITADESTSTAYMELSSLRSEDTAVYYCARGVGYRGVIPLNWFDPWGQGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKGGGKPIPNPLLGLDST

SEQ ID NO: 140 - protein, 237 aa, synthetic construct
METDTLLLWVLLLWVPGSTGDSDDIQMTQTTSSLSASLGDRVTISCRASQDISNYLNWYQQKPDGTVKLLIHYTSRLYSGVPSRFSGSGSGTDYSLTINILEQEDIATYFCQQGDTLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 240 aa, synthetic construct
METDTLLLWVLLLWVPGSTGDSDEIVMMQSPGTLSLSPGERATLSCRASQSVRSNYLAWYQQKPGQAPRLLIYGASSRATGIPDRFSGSGSGTDFTLTISRLEPEDFAVYYCQQYGSSPPRFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 242 aa, synthetic construct
METDTLLLWVLLLWVPGSTGDSDNIMMTQSPSSLAVSAGEKVTMSCKSSQSVLHSSDQKNYLAWYQQKPGQSPKLLIYWASTRESGVPDRFTGSGSGTDFTLTISSVQAEDLAVYFCHQYLSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 243 aa, synthetic construct
METDTLLLWVLLLWVPGSTGDSDDIVMTQSPDSLAVSLGERATINCKSSQSVLASSNNMNYLAWYQQKPGQPPKLLIYWASIRESGVPDRFSGSGPGTDFTLTISSLQTEDVAVYYCHQYYSIPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 239 aa, synthetic construct
METDTLLLWVLLLWVPGSTGDSDEIVLTQSPGILSLSPGERATLSCRASQSVSSSYLAWYQQKPGQAPRLLIYGASSRATGIPDRFSGSGSGTDFTLTISRLEPEDFAVYYCQQYGSSGALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 238 aa, synthetic construct
METDTLLLWVLLLWVPGSTGDSDEIVMTQSPATLSVSPGERATLSCRASQSVSSDLAWYQHKPGQAPRLLIYGASTRATGIPVRFSGSGSGTEFTLTISSLQSEDFAVYYCQQYNNWPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 238 aa, synthetic construct
METDTLLLWVLLLWVPGSTGDSDQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSRMGFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 147 - protein, 129 aa, synthetic construct
QVQLVESGGGVVQPGRSLRLSCAASGLTFSSYVMHWVRQAPGKGLDWVGVIWYDGSKKYYADSVKGRFTISRDNSKNTLYLQMNSLRAEDTAVYYCARDPRDYYDFWSGYDYYYGLDVWGQGTTVTVSS

SEQ ID NO: 148 - protein, 121 aa, synthetic construct
QVQLVQSGAEVKKPGASVKVSCKVSGYTLIELSMHWVRQAPGKGLEWMGGFDPEDVETIYAQQFQGRVTMTEDTSTDTAYMELSSLRSEDTAVYYCATGWAYKSTWYFGYWGQGTLVTVSS

SEQ ID NO: 149 - protein, 124 aa, synthetic construct
QVQLVQSGAEVKKPGSSVKVSCKASGGTFSSYAISWVRQAPGQGLEWMGGNIPIFGTANYAQKFQGRVTITADESTSTAYMELSSLRSEDTAVYYCARGVGYRGVIPLNWFDPWGQGTVVTVSS

SEQ ID NO: 150 - protein, 109 aa, synthetic construct
EIVLTQSPGILSLSPGERATLSCRASQSVSSSYLAWYQQKPGQAPRLLIYGASSRATGIPDRFSGSGSGTDFTLTISRLEPEDFAVYYCQQYGSSGALTFGGGTKVEIK

SEQ ID NO: 151 - protein, 108 aa, synthetic construct
EIVMTQSPATLSVSPGERATLSCRASQSVSSDLAWYQHKPGQAPRLLIYGASTRATGIPVRFSGSGSGTEFTLTISSLQSEDFAVYYCQQYNNWPPFTFGGGTKVEIK

SEQ ID NO: 152 - protein, 109 aa, synthetic construct
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSRMGFGGGTKLTVL

SEQ ID NO: 153 - protein, 107 aa, synthetic construct [sequence omitted]

SEQ ID NO: 154 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 155 - skipped

SEQ ID NO: 156 - protein, 23 aa, synthetic construct
METDTLLLWVLLLWVPGSTGDSD

SEQ ID NO: 157 - protein, 10 aa, synthetic construct
EQKLISEEDL

SEQ ID NO: 158 - protein, 14 aa, synthetic construct
GKPIPNPLLGLDST

SEQ ID NO: 159 - protein, 59 aa, synthetic construct
QRNIRSSRIAGRSALEDLLFSKVVTSGLGTVDVDYKSCTKGLSIADLACAQYYNGIMVL

SEQ ID NO: 160 - protein, 59 aa, synthetic construct
SLPTSGSRVAGRSAIECILFSKLVTSGLGTVDADYKKCTKGLSIADLACAQYYNGIMVL

SEQ ID NO: 161 - protein, 58 aa, synthetic construct
CLGSECSKASSRSAIEDLLFDKVKLSDVGFVEAYNNCTGGAEIRDLICVQSYKGIKVL

SEQ ID NO: 162 - protein, 57 aa, synthetic construct
CLGPHCGSSSRSFFEDLLFDKVKLSDVGFVEAYNNCTGGSEIRDLLCVQSFNGIKVL

SEQ ID NO: 163 - protein, 59 aa, synthetic construct
VSISTGSRSARSAIEDLLFDKVTIADPGYMQGYDDCMQQGPASARDLICAQYVAGYKVL

SEQ ID NO: 164 - protein, 54 aa, synthetic construct
DPLKPTKRSFIEDLLFNKVTLADAGFMKQYGECLGDINARDLICAQKFNGLTVL

SEQ ID NO: 165 - protein, 54 aa, synthetic construct
DPSKPSKRSFIEDLLFNKVTLADAGFIKQYGDCLGDIAARDLICAQKFNGLTVL

SEQ ID NO: 166 - protein, 47 aa, synthetic construct
KPSKRSFIEDLLFNKVTLADAGFIKQYGDCLGDIAARDLICAQKFNG

SEQ ID NO: 167 - protein, 97 aa, synthetic construct
EVKLVESGGGLVKPGGSLKLSCAASGFTFSSYAMSWVRQTPEKRLEWVASISSGGSTYYPDSVKGRFTISRDNARNILYLQMSSLRSEDTAMYYCAR

SEQ ID NO: 168 - protein, 100 aa, synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELP

SEQ ID NO: 169 - protein, 14 aa, synthetic construct
CAREGISVWAMDYW

SEQ ID NO: 170 - protein, 11 aa, synthetic construct
CAQNLELPPTF

SEQ ID NO: 171 - protein, 15 aa, synthetic construct
CARGRDYYGYAMDYW

SEQ ID NO: 172 - protein, 11 aa, synthetic construct
CQHTRELPPTF

SEQ ID NO: 173 - protein, 15 aa, synthetic construct
CASQGIHYYGSFAYW

SEQ ID NO: 174 - protein, 11 aa, synthetic construct [sequence omitted]

SEQ ID NO: 175 - protein, 11 aa, synthetic construct
CAREGNDFDYW

SEQ ID NO: 176 - protein, 11 aa, synthetic construct [sequence omitted]

SEQ ID NO: 177 - protein, 98 aa, synthetic construct
QVQLQQSGPELVRPGVSVKISCKGSGYTFTDYAMHWVKQSHAKSLEWIGVISTYYGDASYNQKFKDKATMTVDKSSSTAYMELARLTSEDSAVYYCAR

SEQ ID NO: 178 - protein, 120 aa, synthetic construct
QVQLQQSGPELVRPGESVKISCKGSGYTFTDYAMHWVKQSHSKSLEWIGIISFYYDNTKYNQNFKGKATMTVDKSSSTAYMELARLTSEDSAIYFCASQGIHYYGSFAYWGQGTLVTVSA

SEQ ID NO: 179 - protein, 120 aa, synthetic construct
QVQLQQSGPELVRPGESVKISCKGSGYTFTDYAMHWVKQSHAKSLEWIGIFSFYYDNTKYNQNFKGKATMTVDKSSSTAYMELARLTSEDSAIYFCASQGIHYYGSFAYWGQGTLVTVSA

SEQ ID NO: 180 - protein, 120 aa, synthetic construct
QVQLQQSGPALVRPGESVKISCKGSGYTFTDYAMHWVKQSRAKSLEWIGIFSFYYDNTNYTQKFKGKATMTVDKSSSTAYMELARLTSEDSAIYYCASQGIHYYGSFAYWGQGTLVTVSA

SEQ ID NO: 181 - protein, 95 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYP

SEQ ID NO: 182 - protein, 107 aa, synthetic construct
DIVMTQSQKFMSTSVGDRVSVTCKASQNVGTNVGWYQQKPGQSHKALIYSASYRYSGVPDRFTGSGSGTDFTLTISNVQSEDLADYFCQQYNNYPLTFGAGTKLELK

SEQ ID NO: 183 - protein, 107 aa, synthetic construct
DIVMTQSQKFMSTSVGDRVSVTCKASQNVGTNVGWYQQKPGQSPQALIYSASYRYSGVPDRFTGSGSGTDFTLTISNVQSEDLAEYFCQQYNSYPLTFGAGTKLELK

SEQ ID NO: 184 - protein, 107 aa, synthetic construct
DIVMTQSQKFMSTSVGDRVSVTCKASQNVATNLGWYQQKPGQSPKTLIYSASYRYSGVPDRFTGSGSGTDFTLTISNVQSEDLAEYFCHQYNGYPLTFGAGTKLELK

(19) United States
(12) Patent Application Publication
(10) Pub. No.: US 2025/0134988 A1
(43) Pub. Date: May 1, 2025

(54) Pan-Coronavirus Immunogenic Compositions

(71) Applicant: The Wistar Institute of Anatomy and Biology, Philadelphia, PA (US)

(72) Inventors: Daniel Kulp, Moorestown, NJ (US); Susanne Walker, Wilmington, DE (US); Kevin Liaw, Haverford, PA (US); Ziyang Xu, Philadelphia, PA (US); David Weiner, Merion, PA (US)

(21) Appl. No.: 18/835,517

(22) PCT Filed: Feb. 3, 2023
(86) PCT No.: PCT/US2023/061940

§ 371 Date: Aug. 2, 2024

Related U.S. Application Data
(60) Provisional application No. 63/306,365, filed on Feb. 3, 2022.

(51) Int. Cl. A61K 39/215; A61P 31/14; C07K 16/10; C12N 7/00; C12N 15/85

(52) U.S. Cl. CPC A61K 39/215; A61P 31/14; C07K 16/1003; C12N 7/00; C12N 15/85; C07K 2317/31; C07K 2317/33; C07K 2317/52; C07K 2317/56; C12N 2770/20022; C12N 2770/20034

(57) ABSTRACT

The present invention generally relates to immunogenic compositions comprising pan-coronavirus antigenic polypeptides and antibodies, and methods of use thereof for the treatment and prevention of coronavirus infection.

### CROSS REFERENCE TO RELATED APPLICATIONS

This application claims priority to U.S. Provisional Application No. 63/306,365, filed Feb. 3, 2022, which is hereby incorporated by reference herein in its entirety.

### STATEMENT REGARDING FEDERALLY SPONSORED RESEARCH OR DEVELOPMENT

This invention was made with government support under AI158777 awarded by the National Institutes of Health. The government has certain rights in the invention.

### BACKGROUND OF THE INVENTION

SARS-CoV-2 has continued to have a global presence with waves of recurrent infections. As public health efforts work to reduce viral transmission and resulting infections, viral variants have surfaced with improved transmission and increased resistance to vaccine-induced immunity. Viral variants can be created through accumulation of mutations. However large exchange of genetic material, or recombination, between SARS-CoV-2 variants has also been observed. There are six other human coronaviruses without approved vaccines and recombination between distinct beta-coronaviruses could easily spark a new pandemic. Approved vaccines elicit neutralizing antibodies to the receptor-binding domain, which is highly variable among the seven human coronaviruses.

Thus, there is a need in the art for improved compositions and methods for the treatment and prevention of coronavirus infection. This invention satisfies this unmet need.

### SUMMARY OF THE INVENTION

In one embodiment, the present invention generally relates to a pan-coronavirus immunogenic composition comprising one or more selected from the group consisting of: a) one or more coronavirus antigenic polypeptide; b) one or more nucleic acid molecule encoding one or more coronavirus antigenic polypeptide; c) one or more coronavirus monoclonal antibody; and d) one or more nucleic acid molecule encoding one or more coronavirus monoclonal antibody.

In one embodiment, said coronavirus antigenic polypeptide of the pan-coronavirus immunogenic composition comprises a coronavirus fusion peptide (FP).

In one embodiment, said coronavirus FP comprises one or more selected from the group consisting of: a) one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114; b) a polypeptide comprising an amino acid sequence at least 90% identical to one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114; c) a polypeptide comprising an amino acid sequence at least 70% of the length of one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114; and d) a polypeptide comprising an amino acid sequence at least 90% identical to and at least 70% the length of one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114.

In one embodiment, said coronavirus antigenic polypeptide comprises a prefusion stabilized spike protein from one or more coronavirus.

In one embodiment, said coronavirus antigenic polypeptide comprises one or more amino acid sequence selected from the group consisting of: SEQ ID NO: 80, SEQ ID NO: 82, SEQ ID NO: 84, SEQ ID NO: 88, SEQ ID NO: 92, SEQ ID NO: 96, SEQ ID NO: 100, SEQ ID NO: 104, SEQ ID NO: 108, SEQ ID NO: 112, SEQ ID NO: 116.

In one embodiment, said one or more coronavirus antigenic polypeptide further comprises a nanoparticle scaffold. In one embodiment, said nanoparticle scaffold comprises at least one of: a GT8-LS-60mer nanoparticle scaffold (SEQ ID NO: 54) and a CA09-FR-24mer nanoparticle scaffold (SEQ ID NO: 68).

In one embodiment, said nucleic acid molecule of the pan-coronavirus immunogenic composition encoding one or more coronavirus antigenic polypeptide comprises one or more selected from the group consisting of: a) one or more nucleotide sequence selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113; b) a nucleotide sequence at least 90% identical to one or more selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113; c) a nucleotide sequence at least 70% of the length of one or more selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113; and d) a nucleotide sequence at least 90% identical to and at least 70% the length of one or more selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113.

In one embodiment, said nucleic acid molecule further comprises a nucleotide sequence encoding a nanoparticle scaffold, wherein said nucleotide sequence is at least one selected from the group consisting of: SEQ ID NO: 53 and SEQ ID NO: 67.

In one embodiment, said nucleic acid molecule comprises a plasmid vector suitable for mammalian expression. In one embodiment, said plasmid vector comprises one or more nucleotide sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47.

In one embodiment, said coronavirus monoclonal antibody of the pan-coronavirus immunogenic composition comprises a heavy chain, and wherein said heavy chain comprises one or more selected from the group consisting of: a) a heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149; b) a polypeptide comprising an amino acid sequence at least 90% identical to one or more heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149; c) a polypeptide comprising an amino acid sequence at least 70% of the length of one or more heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149; and d) a polypeptide comprising an amino acid sequence at least 90% identical to and at least 70% the length of one or more heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149.

In one embodiment, said coronavirus monoclonal antibody of the pan-coronavirus immunogenic composition comprises a light chain, and wherein said light chain comprises one or more selected from the group consisting of: a) a light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152; b) a polypeptide comprising an amino acid sequence at least 90% identical to one or more light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152; c) a polypeptide comprising an amino acid sequence at least 70% of the length of one or more light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152; and d) a polypeptide comprising an amino acid sequence at least 90% identical to and at least 70% the length of one or more light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152.

In one embodiment, said pan-coronavirus immunogenic composition comprises a bispecific antibody comprising a heavy chain IgG Fc “knob”, a heavy chain IgG Fc “hole”, and at least two light chains.

In one embodiment, the pan-coronavirus immunogenic composition comprises one or more coronavirus bispecific monoclonal antibody, wherein: a) said heavy chain IgG Fc “knob” comprises an amino acid sequence selected from the group consisting of: SEQ ID NO: 134, SEQ ID NO: 135, SEQ ID NO: 136, SEQ ID NO: 137, SEQ ID NO: 138, and SEQ ID NO: 139; b) said heavy chain IgG Fc “hole” comprises the amino acid sequence of SEQ ID NO: 133; and c) said at least two light chains comprise at least two amino acid sequences selected from the group consisting of: SEQ ID NO: 140, SEQ ID NO: 141, SEQ ID NO: 142, SEQ ID NO: 143, SEQ ID NO: 144, SEQ ID NO: 145, and SEQ ID NO: 146.

In one embodiment, the present invention generally relates to a method of administering to a subject in need thereof one or more pan-coronavirus immunogenic composition, comprising administering the subject one or more selected from the group consisting of: a) one or more coronavirus antigenic polypeptide; b) one or more nucleic acid molecule encoding one or more coronavirus antigenic polypeptide; c) one or more coronavirus monoclonal antibody; and d) one or more nucleic acid molecule encoding one or more coronavirus monoclonal antibody.

In one embodiment, the present invention generally relates to a method of treating or preventing infection by more than one human coronavirus in a subject, comprising administering to the subject one or more selected from the group consisting of: a) one or more coronavirus antigenic polypeptide; b) one or more nucleic acid molecule encoding one or more coronavirus antigenic polypeptide; c) one or more coronavirus monoclonal antibody; and d) one or more nucleic acid molecule encoding one or more coronavirus monoclonal antibody.

In some embodiments of the methods described herein, said subject is a human. In some embodiments of the methods described herein, the subject is infected with one or more human coronavirus or at risk of becoming infected with one or more human coronavirus. In some embodiments of the methods described herein, the human coronavirus is one or more selected from the group consisting of: 229E, NL63, OC43, HKU1, MERS-CoV, SARS-CoV, and SARS-CoV-2.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIGS. 1A through 1I depict exemplary results demonstrating immunogen design. FIG. 1A depicts the amino acid consensus across the fusion peptide region of human coronaviruses with high conservation shown in the yellow histogram (bottom). FP1, FPlong and FPshort regions of the fusion peptide are highlighted with the antigenic and neutralizing epitopes from previous studies. FIG. 1B depicts the FP epitope region. FIG. 1C depicts an epitope analysis of antibody responses to the Spikeₛₒₗ after autologous immunization. Immunodominance is observed for the RBD epitope while more conserved FP epitope displays a decreased response. FIG. 1D depicts a computational model of FPshort peptide fused to a GT8_LS 60mer nanoparticle scaffold.

FIG. 1E depicts exemplary SEC purification of FPshort nanoparticle showing homogeneous 60mer assembly. FIG. 1F depicts exemplary NS-EM of FPshort nanoparticles displaying spherical nanoparticles in solution. FIG. 1G depicts PDB: 6VSB glycan modeling of the CoV2 spike protein with highlighted 5P prefusion-stabilizing prolines. Green spheres indicate fusion peptide epitope on the prefusion spike protein. FIG. 1H depicts exemplary SEC purification and SEC-MALS analysis of 5P stabilized spike protein in a trimeric homogeneous population. FIG. 1I depicts NS-EM of 6P prefusion-stabilized spike protein as trimeric complexes in solution.

FIGS. 2A through 2H depict exemplary results demonstrating optimization of boosting strategies. FIG. 2A depicts an immunization schedule for BALB/c autologous boosting pilot experiment (top) and exemplary binding titers to FP and Spikeₛₒₗ (bottom). AUC of temporal titers exhibits weak responses to FP when immunizing with Spikeₛₒₗ and mid to high titers for both Spikeₛₒₗ and FP when immunizing with FPshort nanoparticle. FIG. 2B depicts an immunization schedule for BALB/c heterologous boosting FPlong v FPshort experiment (top) and exemplary binding titers to FP and Spikeₛₒₗ (bottom). AUC of temporal titers exhibits weaker responses to FP when immunizing with FPlong/Spikeₛₒₗ and high titers for both Spikeₛₒₗ and FP when immunizing with FPshort/Spikeₛₒₗ. FIG. 2C depicts a computational model of FP glycan shield. Glycan 282 shows the greatest coverage of the FP epitope. FIG. 2D depicts exemplary week 12 serology from pilot experiment of FIG. 2A. Antibody responses are driven towards poorly-folded or post-fusion conformation of spikes from CoV2, CoV1, and MERS but not prefusion-stabilized CoV2 spike proteins 2P or 6P. Recognition of glycan deleted prefusion spike proteins 5P_dG282 and 5P_dGlyMax are observed. FIG. 2E depicts an immunization schedule for BALB/c heterologous boosting experiment with stabilized glycan-deleted spike proteins (top) and exemplary binding titers to FP (bottom). FIG. 2F depicts exemplary pseudovirus neutralization ID₅₀ values from immunized sera showing neutralization 2-4 weeks after heterologous boost with stabilized prefusion spike proteins. FIG. 2G depicts an immunization schedule for C57BL/6 boosting experiment with stabilized glycan-deleted spike proteins (top) and binding titers to FP (bottom). FIG. 2H depicts exemplary pseudovirus neutralization ID₅₀ values from immunized sera showing neutralization 2 weeks after heterologous boost with stabilized prefusion spike proteins but not for the autologous boosting group.

FIGS. 3A through 3E depicts exemplary results demonstrating cross-reactivity. FIG. 3A depicts an exemplary ELISA epitope panel for BALB/c mice humoral response before the heterologous boost (preboost), 2 weeks post-boost, and 4 weeks post-boost. All responses increased post-boost but were not dominated by the typical RBD responses. FIG. 3B depicts an exemplary ELISA pancoronavirus panel for BALB/c mice humoral response 4 weeks after heterologous boost showing an increased cross-reactivity to human coronavirus fusion peptides. FIG. 3C depicts an exemplary ELISA epitope panel for C57BL/6 mice humoral response 2 weeks post-boost. Responses were not dominated by the typical RBD responses and displayed prefusion epitope bias. FIG. 3D depicts an exemplary ELISA pancoronavirus panel for C57BL/6 mice humoral response 2 weeks after heterologous boost showing an increased cross-reactivity to human coronavirus fusion peptides. FIG. 3E depicts an exemplary ELISA against prefusion glycan-deleted spike protein, 5P_dG282, for C57BL/6 mice humoral response before the heterologous boost (preboost), 2 weeks post-boost, and 4 weeks post-boost. All responses increased post-boost.

FIGS. 4A through 4C depict exemplary results demonstrating antibody production. FIG. 4A shows B cell sorting parameters gated for lymphocytes, singlets, live cells, CD19 positive cells, IgM and IgD negative cells, FPshort nanoparticle and 5P_dG282 double-positive population. The combined spleens/lymph nodes were sorted for 4 individual mice and analyzed by 10× for 2 mice individually (mice 101.1 and 101.4) and two mice combined (mice 104.3 and 104.4). FIG. 4B summarizes the BALB/c mice pancoronavirus cross-reactivity and neutralization responses. Circled mice showed exemplary responses and were thus selected for B cell sorting and 10× sequencing. FIG. 4C displays the number of unique clonotypes derived from the 10× sequencing results for each group of sorted and sequenced lymphocytes.

FIGS. 5A through 5C depict exemplary results demonstrating the generation of lineage HV5-6-5/KV2-109 antibodies FIG. 5A displays the lineage tree for paired antibodies recovered from the 10× sequencing under the expanded germline HV5-6-5*01. Highlighted are the antibodies which had exemplary small-scale expression and binding to FP and 5P_dG282. FIG. 5B displays the lineage tree for paired antibodies recovered from the 10× sequencing under the expanded germline KV2-109*01. Highlighted are the antibodies which had exemplary small-scale expression and binding to FP and 5P_dG282. FIG. 5C depicts exemplary binding titers of three antibodies from the small scale expression under the expanded germline HV5-9-5*01 in an ELISA.

FIGS. 6A through 6C depict exemplary results demonstrating lineage HV5-6-5/KV2-109 antibody cross-reactivity and high affinity binding. FIG. 6A depicts an exemplary ELISA for C668 against fusion peptides from human coronaviruses. C668 displays cross-reactivity to SARS CoV2 and SARS CoV1 but not distal human coronaviruses. FIG. 6B depicts SPR results for C668 against CoV2 fusion peptide with kinetics of the interaction shown below. The KD of the antibody-peptide interaction is 50.6 pM. FIG. 6C depicts the AUC from an exemplary ELISA for HV5-6-5/KV2-109 antibodies against prefusion stabilized CoV2 spikes with varying fusion peptide sequences for human coronaviruses and a scrambled CoV2 fusion peptide sequence (5P_NonSFP). C862 shows mild cross-reactivity to other human coronaviruses while C1766 shows increased cross-reactivity to all human coronaviruses. Neither antibodies displayed cross-reactivity to the scrambled fusion peptide sequence.

FIG. 7 depicts a schematic comparison of the C668, C862 and C1766 antibody heavy chains to Mus HV5-6-5. Somatic hypermutations away from germline are shaded. Arrows depict important mutations which could account for increased cross-reactivity to other coronavirus fusion peptide sequences.

FIG. 8 depicts a schematic comparison of the C668, C862 and C1766 antibody light chains to Mus KV2-109. Somatic hypermutations away from germline are shaded. Arrows depict important mutations which could account for increased cross-reactivity to other coronavirus fusion peptide sequences.

FIGS. 9A through 9C depict exemplary results demonstrating the generation of lineage HV1-67/KV6-15 antibodies. FIG. 9A displays the lineage tree for paired antibodies recovered from the 10× sequencing under the expanded germline HV1-67*01. Highlighted are the antibodies which had exemplary small-scale expression and binding to FP and 5P_dG282. FIG. 9B displays the lineage tree for paired antibodies recovered from the 10× sequencing under the expanded germline KV6-15*01. Highlighted are the antibodies which had exemplary small-scale expression and binding to FP and 5P_dG282. FIG. 9C depicts exemplary binding titers of four antibodies from the small scale expression under the expanded germline HV1-67*01 in an ELISA.

FIGS. 10A and 10B depict exemplary results demonstrating lineage HV1-67/KV6-15 antibody cross-reactivity. FIG. 10A depicts an exemplary ELISA for C2691 against fusion peptides from human coronaviruses. C2691 displays cross-reactivity to all human coronaviruses fusion peptides but not an off-target V5 peptide. FIG. 10B depicts the AUC from an exemplary ELISA for HV1-67/KV6-15 antibodies against prefusion stabilized CoV2 spikes with varying fusion peptide sequences for human coronaviruses and a scrambled CoV2 fusion peptide sequence (5P_NonSFP). C2691 and C2503 show high cross-reactivity to other human coronaviruses while C2866 shows slightly decreased cross-reactivity to some human coronaviruses. All antibodies did not display cross-reactivity to the scrambled fusion peptide sequence.

FIGS. 11A and 11B, depict exemplary results demonstrating lineage HV1-67/KV6-15 HCDR3 loop interactions. FIG. 11A depicts exemplary ELISA results for C2691 CDR3 alanine scanning mutations against FP and 5P. A reduction in affinity is observed for antibodies with alanine mutations at positions 107 and 111. Affinity is decreased to a greater extent for the conformational epitope on 5P compared with the linear fusion peptide. FIG. 11B depicts exemplary ELISA results for antibodies with alanine mutations against prefusion stabilized CoV2 spikes with varying fusion peptide sequences for human coronaviruses and a scrambled CoV2 fusion peptide sequence (5P_NonSFP). A reduction in affinity is observed for antibodies with alanine mutations at positions 107 and 111 against all human coronavirus conformational epitopes. No binding is observed against the scrambled fusion peptide spike. A RBD antibody, CR3022, was used as a control for prefusion spike integrity.

FIG. 12 depicts a schematic comparison of the C2691, C2503, C2866 and C2891 antibody heavy chains to Mus HV1-67. Somatic hypermutations away from germline are shaded. Arrows depict important mutations which could account for decreased cross-reactivity to other coronavirus fusion peptide sequences.

FIG. 13 depicts a schematic comparison of the C2691, C2503, C2866 and C2891 antibody light chains to Mus KV6-15. Somatic hypermutations away from germline are shaded. Arrows depict important mutations which could account for decreased cross-reactivity to other coronavirus fusion peptide sequences.

FIGS. 14A through 14D depict exemplary results demonstrating that both the heavy chain and the light chain for antibodies C668 and C2691 are important for binding. FIG. 14A depicts the differences in heavy chain and light chain paring and CDR3 sequences between functional (light grey) and non-functional (dark grey) antibodies from the two expanded lineages. FIG. 14B depicts an exemplary ELISA against 5P_dG282 for small-scale expression and binding of paired heavy and light chains or swapped heavy chains or light chains for the two antibody lineages represented with C668 and C2691 antibodies. Only the paired antibodies display functional binding to 5P_dG282. FIG. 14C depicts an exemplary ELISA against FP for small-scale expression and binding of paired heavy and light chains or swapped heavy chains or light chains for the two antibody lineages represented with C668 and C2691 antibodies. Only the paired antibodies display functional binding to FP. FIG. 14D depicts a simple western blot detecting Fc expression from the small scale cultures by chemiluminescence. All antibodies with swapped heavy and light chain pairing displayed expression except for C2944 HC/C2691 LC pairing.

FIGS. 15A and 15B depict exemplary results demonstrating that position 13 on CoV2 fusion peptide can knock down antibody binding. FIG. 15A depicts the 5P stabilized prefusion spikes with mutations at position 13 or 15 to include a large amino acid substitution (tryptophan) or a glycan substitution (NXT motif) at position 13. FIG. 15B depicts an exemplary ELISA for antibodies C668 and C2691 and control antibody CR3022 and receptor ACE2 with an Fc tag against the knockdown spike, 5P_KO1, or 5P. CR3022 and ACE2 retain binding to 5P_KO1 while C668 and C2691 display decreased affinity for the knockdown mutation (solid lines) compared to 5P (dashed lines).

FIGS. 16A through 16D depict exemplary results demonstrating that multiple glycan deletions aid in antibody binding. FIG. 16A depicts exemplary ELISA binding curves for C2691 binding against 5P and 5P with glycan deletions. Single glycan deletions were examined at positions 282, 603, 616, and 801 and multiple glycan deletions were examined for impacts to affinity using a triple glycan deletion spike composed of deletions at positions 282, 603 and 616 (5P_FPdG3). The single deletion at position 603 had a mild impact of antibody binding while 5P_FPdG3 showed greater improvement to antibody binding. FIG. 16B depicts the EC50 ratio of C2691binding to the glycan deleted spike over the wildtype 5P. Bars below 10⁰ indicate increased affinity while bars above 100 indicate decreased affinity. Deletion of multiple glycans has the greatest impact on increased antibody affinity. 6P contains a proline in the fusion peptide region and abolishes binding of C2691 to the conformational epitope. FIG. 16C depicts exemplary ELISA binding curves for C668 binding against 5P and 5P with glycan deletions. The single deletion at position 603 had a mild impact of antibody binding while 5P_FPdG3 showed greater improvement to antibody binding. FIG. 16D depicts the EC50 ratio of C668 binding to the glycan deleted spike over the wildtype 5P. Deletion of multiple glycans has the greatest impact on increased antibody affinity. 6P contains a proline in the fusion peptide region and abolishes binding of C668 to the conformational epitope.

### DETAILED DESCRIPTION

The present invention generally relates to novel pan-coronavirus immunogenic compositions for the treatment or prevention of coronavirus infection. The invention is based, in part, upon a sequence analysis of the seven known human coronaviruses, which identified a highly conserved fusion peptide (FP) region. The present invention is further based, in part, upon the design and construction of a novel FP-based immunogenic composition to elicit broad reactivity across the known human coronaviruses and the generation of monoclonal antibodies therefrom.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F (ab) 2, as well as single chain antibodies (scFv), heavy chain antibodies, such as camelid antibodies, synthetic antibodies, chimeric antibodies, and a humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

“Antigen” or “antigenic polypeptide”, as used interchangeably herein, refers to proteins that have the ability to generate an immune response in a host. An antigen may be recognized and bound by an antibody. An antigen may originate from within the body or from the external environment.

The term “bispecific”, as used herein, refers to agents (e.g., ligands or antibodies) that recognize two different antigens by virtue of possessing at least one region (e.g., a ligand or a Fab of a first antibody) that is specific for a first target, and at least a second region (e.g., a ligand or a Fab of a second antibody) that is specific for a second target.

A “consensus sequence”, as used herein, refers to a sequence derived from a sequence alignment of more than one variable sequence. For example, a calculated order of the most frequent residues in the alignment of multiple amino acid sequences of an antigen can be used to define a consensus sequence for that antigen.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Fragment”, as used herein in reference to the polypeptides and nucleic acid molecules of the present invention, refers to a portion of the full-length amino acid or nucleotide sequence. For example, a fragment may be a polypeptide or nucleic acid molecule that is 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of the reference amino acid or nucleotide sequence. In some embodiments, the fragment is 100% identical to the portion of the reference sequence from which is derives. An “immunogenic fragment” refers to a fragment of an antigenic polypeptide or nucleic acid molecule encoding an antigenic polypeptide that induces a detectable level of immunogenicity relative to the reference polypeptide or nucleic acid molecule.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous”, “identical,” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response,” as the term is used herein, means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

The term “immunogen” or “immunogenic” as used herein, is intended to denote a substance of matter, which is capable of inducing an adaptive immune response in an individual, where said adaptive immune response is capable of inducing an immune response which significantly engages pathogenic agents, which share immunological features with the immunogen. “Immunogen” refers to any substance introduced into the body in order to generate an immune response. That substance can a physical molecule, such as a protein, or can be encoded by a vector, such as DNA, mRNA, or a virus.

A “nucleic acid” or “nucleic acid molecule” refers to a polynucleotide and includes poly-ribonucleotides and poly-deoxyribonucleotides. Nucleic acids according to the present invention may include any polymer or oligomer of pyrimidine and purine bases, such as cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The terms “patient,” “subject,” “individual,” and the like are used interchangeably herein, and refer to any animal, or cells thereof whether in vivo, in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, mutant polypeptides, variant polypeptides, or a combination thereof.

To “prevent” a disease or disorder as the term is used herein, means to reduce the severity or frequency of at least one sign or symptom of a disease or disorder that is to be experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

The term “therapeutically effective amount” refers to the amount of the subject compound or composition that will elicit the biological, physiologic, clinical or medical response of a cell, tissue, organ, system, or subject that is being sought by the researcher, veterinarian, medical doctor or other clinician. The term “therapeutically effective amount” includes that amount of a compound or composition that, when administered, is sufficient to prevent development of, or treat to some extent, one or more of the signs or symptoms of the disorder or disease being treated. The therapeutically effective amount will vary depending on the compound or composition, the disease and its severity and the age, weight, etc., of the subject to be treated.

To “treat” a disease or disorder as the term is used herein, means to reduce the frequency or severity of at least one sign or symptom of a disease or disorder experienced by a subject. The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes:
(a) preventing the disease and/or symptom(s) from occurring in a subject who may be predisposed to the disease or symptom but has not yet been diagnosed as having it;
(b) inhibiting the disease and/or symptom(s), e.g., slowing or arresting their development;
or (c) relieving the disease symptom(s), i.e., causing regression of the disease and/or symptom(s). Those in need of treatment include those already inflicted as well as those in which prevention is desired.

“Variant”, as used herein in reference to polypeptides and nucleic acid molecules of the present invention, refers to a change in amino acid or nucleotide sequence relative to a reference sequence and includes translocations, deletions, insertions, and substitutions/point mutations. For example, a variant may be 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to the reference amino acid or nucleotide sequence. An “immunogenic variant” refers to a variant of an antigenic polypeptide or nucleic acid molecule encoding an antigenic polypeptide that induces a detectable level of immunogenicity relative to the reference polypeptide or nucleic acid molecule.

### Compositions

In one embodiment, the present invention comprises a pan-coronavirus immunogenic composition. In one embodiment, the composition comprises one or more coronavirus antigenic polypeptide. In one embodiment, the composition comprises one or more nucleic acid molecule encoding one or more coronavirus antigenic polypeptide. In one embodiment, the composition comprises one or more coronavirus monoclonal antibody. In one embodiment, the composition comprises one or more nucleic acid molecule encoding one or more coronavirus monoclonal antibody.

### Antigenic Polypeptides

In one embodiment, the present invention comprises a pan-coronavirus immunogenic composition comprising one or more coronavirus antigenic polypeptide. In one embodiment, the antigenic polypeptide comprises a coronavirus polypeptide corresponding to an amino acid sequence that is highly conserved across more than one coronavirus. In one embodiment, the antigenic polypeptide comprises a coronavirus polypeptide corresponding to an amino acid sequence that is highly conserved across more than one human coronavirus. In one embodiment, said more than one human coronavirus comprises at least two human coronaviruses. In one embodiment, said more than one human coronavirus comprises at least three human coronaviruses. In one embodiment, said more than one human coronavirus comprises at least four human coronaviruses. In one embodiment, said more than one human coronavirus comprises at least three human coronaviruses. In one embodiment, said more than one human coronavirus comprises at least five human coronaviruses. In one embodiment, said more than one human coronavirus comprises at least six human coronaviruses. In one embodiment, said more than one human coronavirus comprises at least seven human coronaviruses. In one embodiment, human coronaviruses include, but are not limited to, 229E, NL63, OC43, HKU1, MERS-CoV, SARS-CoV, and SARS-CoV-2.

In one embodiment, the antigenic polypeptide comprises a coronavirus polypeptide corresponding to an amino acid sequence from one or more coronavirus spike protein. In one embodiment, the coronavirus polypeptide corresponding to an amino acid sequence from one or more coronavirus spike protein comprises the fusion peptide (FP) of the spike protein.

In one embodiment, the antigenic polypeptide comprises a consensus antigenic polypeptide. In one embodiment, the consensus antigenic polypeptide comprises a consensus coronavirus polypeptide corresponding to an amino acid sequence from one or more coronavirus spike protein. In one embodiment, the consensus coronavirus polypeptide comprises a consensus fusion peptide (FP) of the spike protein.

In some embodiments, the antigenic polypeptide comprising an FP or a consensus FP of the spike protein of one or more coronavirus comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to one or more sequence selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114.

In one embodiment, the antigenic polypeptide comprising an FP or a consensus FP of the spike protein of one or more coronavirus comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114.

In one embodiment, the antigenic polypeptide comprising an FP or a consensus FP of the spike protein of one or more coronavirus comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to and 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114.

In one embodiment, the antigenic polypeptide comprising an FP or a consensus FP of the spike protein of one or more coronavirus comprises an amino acid sequence one or more sequence selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114.

In one embodiment, the antigenic polypeptide of the pan-coronavirus immunogenic composition comprises a prefusion stabilized spike protein from one or more coronavirus. In one embodiment, the FP or consensus FP of the present invention replaces the endogenous FP of the prefusion stabilized spike protein from one or more coronavirus. In one embodiment, said one or more coronavirus comprises one or more human coronavirus. In some embodiments, human coronaviruses include, but are not limited to, 229E, NL63, OC43, HKU1, MERS-CoV, SARS-CoV, and SARS-CoV-2. In one embodiment, said one or more human coronavirus comprises SARS-CoV-2. In one embodiment, the antigenic polypeptide of the pan-coronavirus immunogenic composition comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to one or more sequence selected from the group consisting of: SEQ ID NO: 80, SEQ ID NO: 82, SEQ ID NO: 84, SEQ ID NO: 88, SEQ ID NO: 92, SEQ ID NO: 96, SEQ ID NO: 100, SEQ ID NO: 104, SEQ ID NO: 108, SEQ ID NO: 112, SEQ ID NO: 116.

In one embodiment, the antigenic polypeptide of the pan-coronavirus immunogenic composition comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 80, SEQ ID NO: 82, SEQ ID NO: 84, SEQ ID NO:

88, SEQ ID NO: 92, SEQ ID NO: 96, SEQ ID NO: 100, SEQ ID NO: 104, SEQ ID NO: 108, SEQ ID NO: 112, SEQ ID NO: 116.

In one embodiment, the antigenic polypeptide of the pan-coronavirus immunogenic composition comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to and 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 80, SEQ ID NO: 82, SEQ ID NO: 84, SEQ ID NO: 88, SEQ ID NO: 92, SEQ ID NO: 96, SEQ ID NO: 100, SEQ ID NO: 104, SEQ ID NO: 108, SEQ ID NO: 112, SEQ ID NO: 116.

In one embodiment, the antigenic polypeptide of the pan-coronavirus immunogenic composition comprises an amino acid sequence selected from the group consisting of: SEQ ID NO: 80, SEQ ID NO: 82, SEQ ID NO: 84, SEQ ID NO: 88, SEQ ID NO: 92, SEQ ID NO: 96, SEQ ID NO: 100, SEQ ID NO: 104, SEQ ID NO: 108, SEQ ID NO: 112, SEQ ID NO: 116.

One of skill in the art will recognize that any known methods of producing polypeptides can be used to generate the polypeptide(s) of the present invention. The polypeptide(s) of the present invention may be made using chemical methods. For example, polypeptide(s) can be synthesized by solid phase techniques (Roberge J Y et al (1995) Science 269:202-204), cleaved from the resin, and purified by preparative high-performance liquid chromatography. Automated synthesis may be achieved, for example, using the ABI 431 A Peptide Synthesizer (Perkin Elmer) in accordance with the instructions provided by the manufacturer.

Polypeptide(s) of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

The polypeptide(s) may alternatively be made by recombinant means or by cleavage from one or more longer polypeptide(s). The composition of the polypeptide(s) may be confirmed by amino acid analysis or sequencing.

The polypeptide(s) of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptide(s) of the invention may include unnatural amino acids formed by post-translational modification or by introducing unnatural amino acids during translation. A variety of approaches are available for introducing unnatural amino acids during protein translation.

The polypeptide(s) of the invention may be phosphorylated using conventional methods such as the method described in Reedijk et al. (The EMBO Journal 11 (4): 1365, 1992).

The polypeptide(s) of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Nanoparticles

In one embodiment, the antigenic polypeptide of the present invention further comprises a nanoparticle scaffold. In one embodiment, the nanoparticle scaffold is N-terminally conjugated to the antigenic polypeptide. In one embodiment, the nanoparticle scaffold is C-terminally conjugated to the antigenic polypeptide. In one embodiment, the nanoparticle scaffold comprises an amino acid sequence that self-assembles into a multimeric nanoparticle. In one embodiment, the nanoparticle scaffold comprises an amino acid sequence that self-assembles into a 60mer nanoparticle. In one embodiment, the nanoparticle scaffold comprises a GT8-LS-60mer nanoparticle scaffold. In one embodiment, the GT8-LS-60mer nanoparticle scaffold comprises the amino acid sequence of SEQ ID NO: 54. In one embodiment, the GT8-LS-60mer nanoparticle scaffold is encoded by the nucleotide sequence of SEQ ID NO: 53. In one embodiment, the nanoparticle scaffold comprises an amino acid sequence that self-assembles into a 24mer nanoparticle. In one embodiment, the nanoparticle scaffold comprises a CA09-FR-24mer nanoparticle scaffold. In one embodiment, the CA09-FR-24mer nanoparticle scaffold comprises the amino acid sequence of SEQ ID NO: 68. In one embodiment, the CA09-FR-24mer nanoparticle scaffold is encoded by the nucleotide sequence of SEQ ID NO: 67.

In one embodiment, the nanoparticle scaffold is separated from the antigenic polypeptide by a linker. In one embodiment, the linker comprises glycine and serine residues. In one embodiment, the linker comprises the amino acid sequence of SEQ ID NO: 52. In one embodiment, the linker is encoded by the nucleotide sequence of SEQ ID NO: 51.

### Vaccines

In some embodiments, the pan-coronavirus immunogenic composition of the present invention comprises a vaccine. For a composition to be useful as a vaccine, the composition must induce an immune response against one or more coronavirus antigen in a cell, tissue or subject. In some embodiments, the composition induces an immune response against one or more consensus coronavirus antigen in a cell, tissue or subject. In some instances, the vaccine induces a protective immune response in the subject. In some instances, the vaccine induces a protective immune response against more than one human coronavirus in the subject.

In some embodiments, the present disclosure comprises pan-coronavirus nucleic acid vaccine. Nucleic acid-based vaccines are known to elicit a prominent cell-mediated immune response. See. e.g., Donnely et al., 1997; Rosenberg, S. A., Immunity 10:281, 1999. Thus, the antigenic agent for use in the vaccines of the disclosure can take the form of a polynucleotide that can stimulate an immune response against one or more coronavirus antigenic polypeptide, variant or a fragment thereof when administered to a subject.

The form of the nucleic acid used in a vaccine of the disclosure can be any suitable for stimulating an immune response against one or more coronavirus when administered to a subject. For example, the nucleic acid can be in the form of “naked DNA” or it can be incorporated in an expression vector. A description of suitable nucleic acids is presented below. Nucleic acids that are most immunogenic in a subject can be determined by preparing several of the below listed nucleic acids (e.g., those that encode the whole antigen, variants or peptide fragments thereof), administering to the subject (or a series of genetically similar such subjects) such nucleic acids in a vaccine composition (e.g., as naked nucleic acid or in an expression vector in a suitable carrier), and analyzing the subject(s) for the stimulation of an immune response. Those nucleic acids that induce the desired response can then be selected.

The disclosure provides for the use of nucleic acid vaccines to stimulate an immune response against one or more coronavirus in a subject. The use of nucleic acids for stimulating both class I and class II restricted immune responses against a particular protein is known in the art. See. e.g., Rosenberg, S. A., Immunity 10:281, 1999; Ulmer et al., Science, 259:1745, 1993; Donnelly et al., Ann. NY Acad. Sci., 772:40, 1995; Scheurs et al., Cancer Res. 58:2509, 1998; Hurpin et al., Vaccine 16:208, 1998; Lekutis et al., J. Immunol. 158:4471, 1997; Manickan et al., J. Leukoc. Biol. 61:125, 1997. Nucleic acid vaccines can be administered to a subject by any suitable technique. For example, naked DNA can be injected into muscle cells of a subject or naked DNA-coated gold particles can be introduced into skin cells (to be taken up by dendritic cells) of a subject using a gene gun.

### Monoclonal Antibodies

In one embodiment, the pan-coronavirus immunogenic composition of the present invention comprises one or more antibody. In one embodiment, the antibody comprises a monoclonal antibody. In some embodiments, the monoclonal antibody comprises a heavy chain and a light chain.

In some embodiments, the heavy chain comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to one or more sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149.

In some embodiments, the heavy chain comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149.

In some embodiments, the heavy chain comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to and 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149.

In some embodiments, the heavy chain comprises one or more amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149.

In some embodiments, the light chain comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to one or more sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152.

In some embodiments, the light chain comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152.

In some embodiments, the light chain comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to and 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152.

In some embodiments, the light chain comprises one or more amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152.

In some embodiments, the monoclonal antibody of the pan-coronavirus immunogenic composition comprises a bispecific antibody. In one embodiment, said bispecific antibody comprises a heavy chain IgG Fc “knob” and a heavy chain IgG Fc “hole”. One of skill in the art will recognize that a heavy chain IgG “knob” specific to one antigen will heterodimerize with the corresponding IgG Fc “hole” specific to a separate antigen, thereby generating a bispecific heavy chain directed to two separate antigens. In one embodiment, the IgG Fc “knob” and IgG Fc “hole” are variants of IgG Fc. In one embodiment, said IgG Fc comprises human IgG Fc. In one embodiment, said human IgG Fc comprises human IgG1 Fc.

In one embodiment, the heavy chain IgG Fc “knob” comprises an amino acid sequence selected from the group consisting of: SEQ ID NO: 134, SEQ ID NO: 135, SEQ ID NO: 136, SEQ ID NO: 137, SEQ ID NO: 138, and SEQ ID NO: 139, or a variant or fragment thereof.

In one embodiment, the heavy chain IgG Fc “hole” comprises the amino acid sequence of SEQ ID NO: 133, or a variant or fragment thereof.

In some embodiments, the bispecific antibody further comprises one or more antibody light chain. In one embodiment, the light chain comprises an amino acid sequence selected from the group consisting of: SEQ ID NO: 140, SEQ ID NO: 141, SEQ ID NO: 142, SEQ ID NO: 143, SEQ ID NO: 144, SEQ ID NO: 145, and SEQ ID NO: 146. In one embodiment, the bispecific antibody comprises at least two different antibody light chains. In one embodiment, said at least two antibody light chains comprise at least two different amino acid sequences selected from the group consisting of: SEQ ID NO: 140, SEQ ID NO: 141, SEQ ID NO: 142, SEQ ID NO: 143, SEQ ID NO: 144, SEQ ID NO: 145, and SEQ ID NO: 146.

### Additional Sequences

In some embodiments, the antigenic polypeptide or monoclonal antibody of the present invention comprises one or more purification tag. In one embodiment, the purification tag comprises a polypeptide derived from the c-myc gene product (i.e., myc tag). In one embodiment, the myc tag comprises an amino acid sequence of SEQ ID NO: 157. In one embodiment, the purification tag comprises a polypeptide derived from the P and V protein of the simian virus 5 (i.e., V5 tag). In one embodiment, the V5 tag comprises an amino acid sequence of SEQ ID NO: 158.

In some embodiments, the antigenic polypeptide or monoclonal antibody of the present invention further comprises a signal peptide. In one embodiment, the signal peptide promotes extracellular secretion of the antigenic polypeptide or monoclonal antibody. In some embodiments, the signal peptide comprises an amino acid sequence selected from the group consisting of: SEQ ID NO: 50 and SEQ ID NO: 156.

### Nucleic Acids

In one embodiment, the pan-coronavirus immunogenic composition of the present invention comprises one or more nucleic acid molecule encoding one or more coronavirus antigenic polypeptide.

In one embodiment, the pan-coronavirus immunogenic composition of the present invention comprises one or more nucleic acid molecule encoding one or more coronavirus antigenic polypeptide.

In one embodiment, the nucleic acid molecule encoding the coronavirus antigenic polypeptide comprises a nucleotide sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to one or more sequence selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113.

In one embodiment, the nucleic acid molecule encoding the coronavirus antigenic polypeptide comprises a nucleotide sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113.

In one embodiment, the nucleic acid molecule encoding the coronavirus antigenic polypeptide comprises a nucleotide sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to and 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of one or more sequence selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113.

In one embodiment, the nucleic acid molecule encoding the coronavirus antigenic polypeptide comprises one or more nucleotide sequence selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113.

In one embodiment, the pan-coronavirus immunogenic composition of the present invention comprises one or more nucleic acid molecule encoding one or more monoclonal antibody.

In one embodiment, the nucleic acid molecule encoding the monoclonal antibody comprises a nucleotide sequence encoding a heavy chain selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149, or a variant or fragment thereof.

In one embodiment, the nucleic acid molecule encoding the monoclonal antibody comprises a nucleotide sequence encoding a light chain selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152, or a variant or fragment thereof.

In one embodiment, the nucleic acid molecule encoding the monoclonal antibody comprises a nucleotide sequence encoding: 1) a heavy chain selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149, or a variant or fragment thereof; and b) a light chain selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152, or a variant or fragment thereof.

In one embodiment, the nucleic acid molecule encoding the monoclonal antibody comprises a nucleotide sequence encoding: a) a heavy chain IgG Fc “knob” comprising an amino acid sequence selected from the group consisting of: SEQ ID NO: 134, SEQ ID NO: 135, SEQ ID NO: 136, SEQ ID NO: 137, SEQ ID NO: 138, and SEQ ID NO: 139, or a variant or fragment thereof; b) a heavy chain IgG Fc “hole” comprising the amino acid sequence of SEQ ID NO: 133, or a variant or fragment thereof; and c) at least two different amino acid sequences selected from the group consisting of: SEQ ID NO: 140, SEQ ID NO: 141, SEQ ID NO: 142, SEQ ID NO: 143, SEQ ID NO: 144, SEQ ID NO: 145, and SEQ ID NO: 146, or variants or fragments thereof.

The nucleic acid molecule(s) encoding the polypeptide(s) of the present invention can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The nucleic acid molecule(s) may comprise any type of nucleic acid, including, but not limited to DNA and RNA. For example, in one embodiment, the composition comprises an isolated DNA molecule, including for example, an isolated cDNA molecule, encoding the polypeptide(s) of the invention. In one embodiment, the composition comprises an isolated RNA molecule encoding the polypeptide(s) of the invention, or a functional fragment thereof.

In some embodiments, the nucleic acid molecule further comprises an expression vector. The expression of natural or synthetic nucleic acids encoding a polypeptide of the invention is typically achieved by operably linking a nucleic acid encoding the fusion protein of the invention or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses (AAV), herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, the expression vector is a plasmid vector. In one embodiment, the plasmid vector comprises a vector designed for expression in mammalian cells. In one embodiment, the plasmid vector comprises a vector designed for DNA vaccines. In one embodiment, the DNA vaccine plasmid vector comprises a pVAX™ vector. In one embodiment, the plasmid vector comprises a heavy chain variable region cloning plasmid that expresses the constant region of the human IgG1 heavy chain and the IL2 signal sequence. In one embodiment, the plasmid vector comprises a pFUSEss-CHIg-hG1 vector. In one embodiment, the plasmid vector comprises a light chain variable region cloning plasmid that expresses the constant region of the human kappa light chain and the IL2 signal sequence. In one embodiment, the plasmid vector comprises a pFUSE2ss-CLIg-hK vector. In one embodiment, the plasmid vector comprises a light chain variable region cloning plasmid that expresses the constant region of the human lambda 2 light chain and the IL2 signal sequence. In one embodiment, the plasmid vector comprises a pFUSE2ss-CLIg-hL2 vector.

### Methods of Administration, Treatment and Prevention

In one embodiment, the present invention comprises a method of administering to a subject in need thereof one or more pan-coronavirus immunogenic composition. In one embodiment, the method comprises administering to the subject one or more selected from the group consisting of: a) one or more coronavirus antigenic polypeptide of the present invention; b) one or more nucleic acid molecule encoding one or more coronavirus antigenic polypeptide of the present invention; c) one or more coronavirus monoclonal antibody of the present invention; and d) one or more nucleic acid molecule encoding one or more coronavirus monoclonal antibody of the present invention.

In one embodiment, the invention comprises a method of inducing an immune response in a subject comprising administering to the subject: a) one or more coronavirus antigenic polypeptide of the present invention; b) one or more nucleic acid molecule encoding one or more coronavirus antigenic polypeptide of the present invention; c) one or more coronavirus monoclonal antibody of the present invention; and d) one or more nucleic acid molecule encoding one or more coronavirus monoclonal antibody of the present invention.

In one embodiment, the invention comprises a method of treating or preventing infection by at least one human coronavirus in a subject. In some embodiments, the invention comprises a method of treating of preventing more than one human coronavirus in a subject. In one embodiment, the method comprise administering to the subject: a) one or more coronavirus antigenic polypeptide of the present invention; b) one or more nucleic acid molecule encoding one or more coronavirus antigenic polypeptide of the present invention; c) one or more coronavirus monoclonal antibody of the present invention; and d) one or more nucleic acid molecule encoding one or more coronavirus monoclonal antibody of the present invention.

In some embodiments, the subject is a mammal. In one embodiment, the subject is a human. In some embodiments, the subject is infected with one or more human coronavirus. In some embodiments, the subject is at risk of becoming infected with one or more human coronavirus. In some embodiments, the human coronavirus is one or more selected from the group consisting of: 229E, NL63, OC43, HKU1, MERS-CoV, SARS-CoV, and SARS-CoV-2.

In some embodiments, said one or more coronavirus antigenic polypeptide comprises one or more amino acid sequence selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114, or a variant or fragment thereof. In some embodiments, said one or more nucleic acid molecule encoding one or more coronavirus antigenic polypeptide comprises one or more nucleotide sequence selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113, or a variant or fragment thereof.

In some embodiments, said one or more coronavirus monoclonal antibody comprises: a) at least one heavy chain comprising an amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149, or a variant or fragment thereof; and b) at least one light chain comprising an amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152, or a variant or fragment thereof.

In some embodiments, said one or more nucleic acid molecule encoding one or more coronavirus monoclonal antibody comprises at least one nucleotide sequence selected from the group consisting of: a) a nucleotide sequence encoding at least one heavy chain comprising an amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149, or a variant or fragment thereof; and b) a nucleotide sequence encoding at least one light chain comprising an amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152, or a variant or fragment thereof.

Administration of the compositions of the disclosure in a method of treatment can be achieved in a number of different ways, using methods known in the art. In one embodiment, the method of the disclosure comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The therapeutic and prophylactic methods of the disclosure further encompass the use of pharmaceutical compositions comprising and/or encoding an antigenic polypeptide or monoclonal antibody described herein to practice the methods of the disclosure. The pharmaceutical compositions useful for practicing the invention may be administered to deliver a dose of between 1 ng/kg/day and 100 mg/kg/day. In one embodiment, the invention envisions administration of a dose which results in a concentration of the compound of the present disclosure from 10 nM and 10 μM in a mammal.

Typically, dosages which may be administered in a method of the disclosure to a mammal, for example a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The present invention encompasses the preparation and use of pharmaceutical compositions for administration comprising a composition of the present invention, disclosed herein, as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art. In various embodiments, the active ingredient is one or more nucleic acid molecule, one or more polypeptide, or a combination thereof, as elsewhere described herein.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, other preparations containing the active ingredient, and immunologically based systems may also be used to administer an appropriate modulator thereof, according to the methods of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Typical dosages of the composition of the invention which may be administered to an animal (e.g. a human) range in amount from about 0.001 mg to about 1000 mg per kilogram of body weight of the subject. The precise dosage administered will vary depending upon any number of factors, including, but not limited to, the type of animal and type of disease or disorder being treated, the age of the animal and the route of administration. In some embodiments, the dosage of the compound will vary from about 0.1 mg to about 10 mg per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease or disorder being treated, the type and age of the animal, etc.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Pan-Coronavirus Immunogen Design

The present Example demonstrates the development of a novel nanoparticle-spike vaccine regime that targets the fusion peptide (FP) epitope, which is highly conserved across the seven known human coronaviruses.

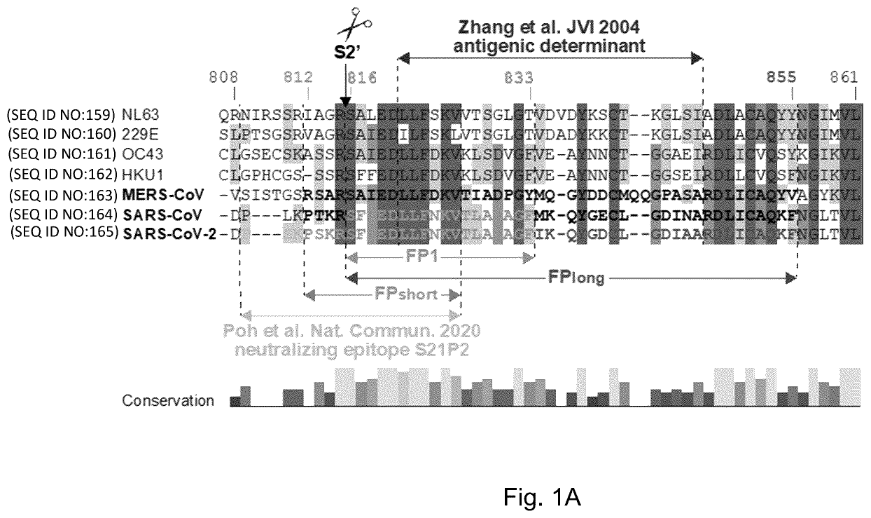

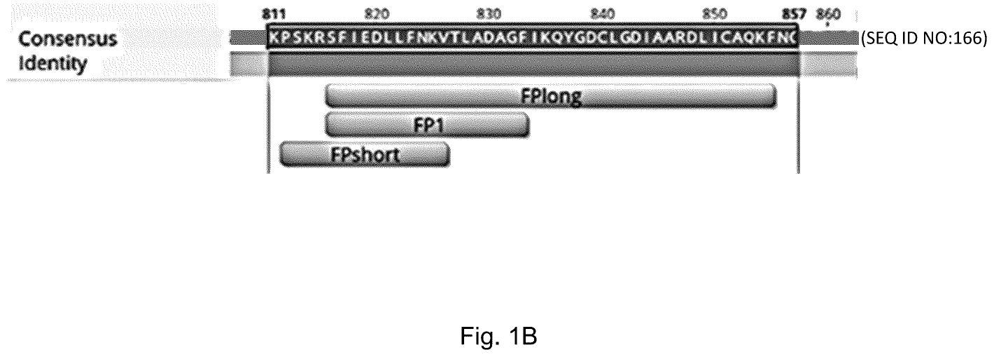

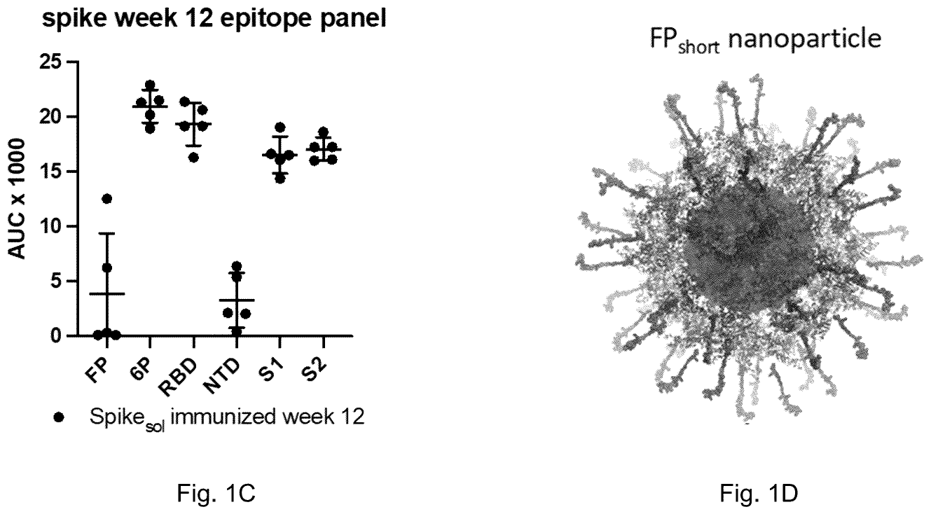

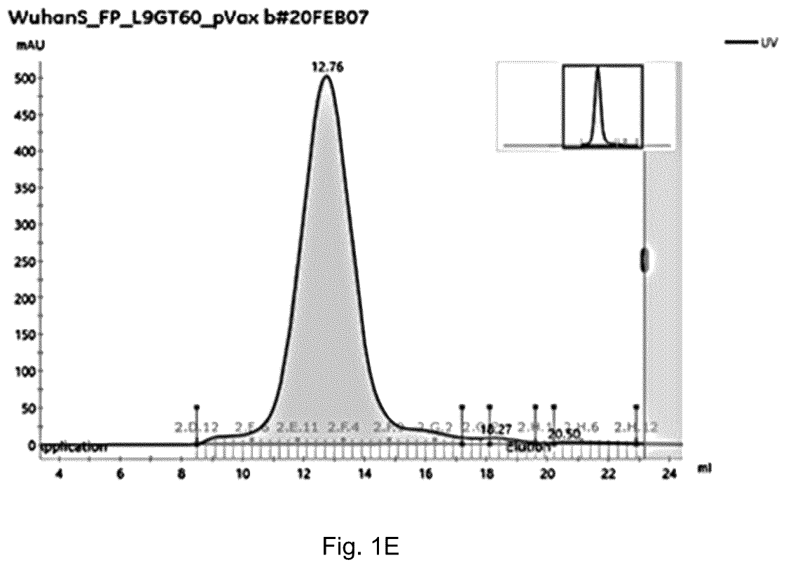

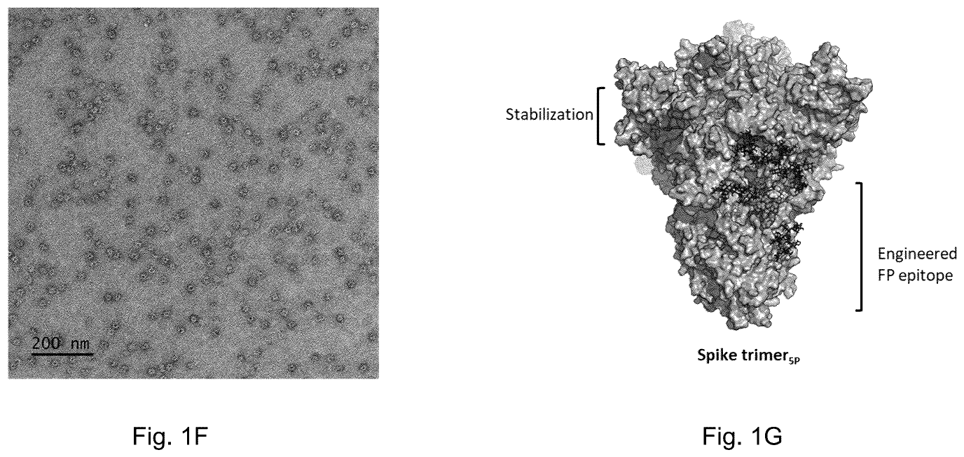

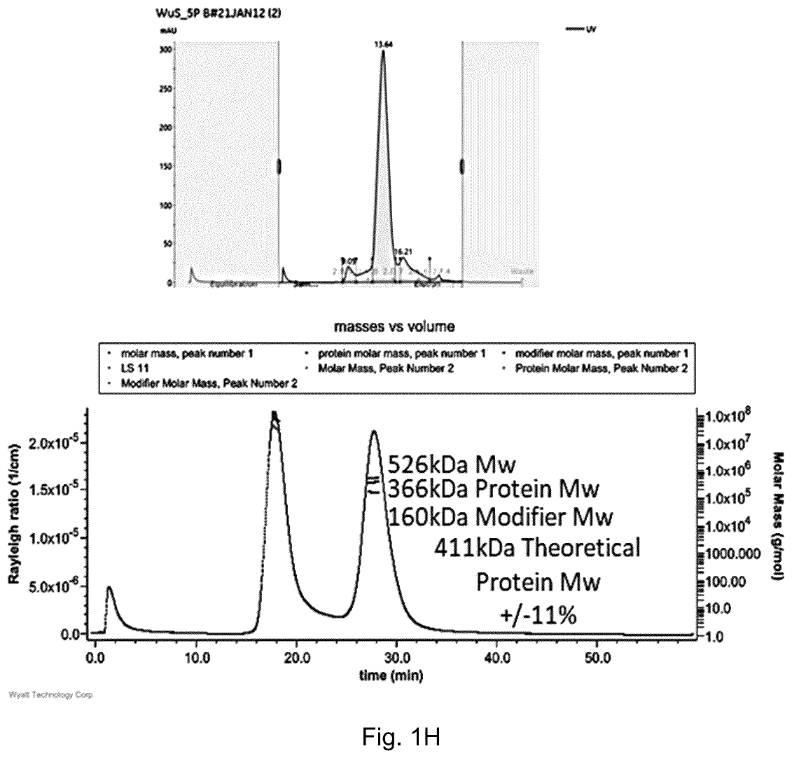

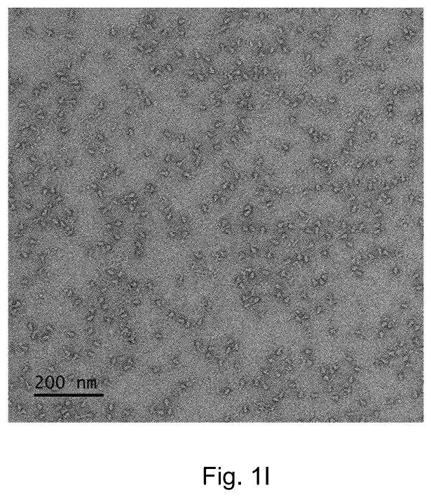

FIG. 1 highlights the pan-coronavirus immunogen design, based on the highly conserved fusion peptide of the human coronavirus spike protein. FIG. 1A depicts a sequence alignment of the fusion peptide region for all coronaviruses that infect humans. A CoV2 mutational analysis of the fusion peptide and RBD epitopes identified conservation of the FP epitope throughout variants of concern for the global pandemic, in contrast to the highly variable RBD epitope. FIG. 1B shows the fusion peptide consensus sequence. FIG. 1C shows immunogenicity as Area-Under-the-Curve (AUC) analysis as determined by ELISA against SARS-CoV-2 spike protein epitopes after immunization with a standard full length spike vaccine. FIG. 1D depicts a structural model of the fusion peptide nanoparticle. FIG. 1E depicts a size exclusion chromatogram showing homogeneity and assembly of the nanoparticle. FIG. 1F depicts negative-stain electron microscopy images of the fusion peptide nanoparticle showing assembly. FIG. 1G depicts a structural model of the 5P trimer immunogen. FIG. 1H depicts the biophysical data (size-exclusion and multi-angle light scattering) for the 5P trimer. FIG. 1I depicts negative-stain electron microscopy data of the 5P trimer.

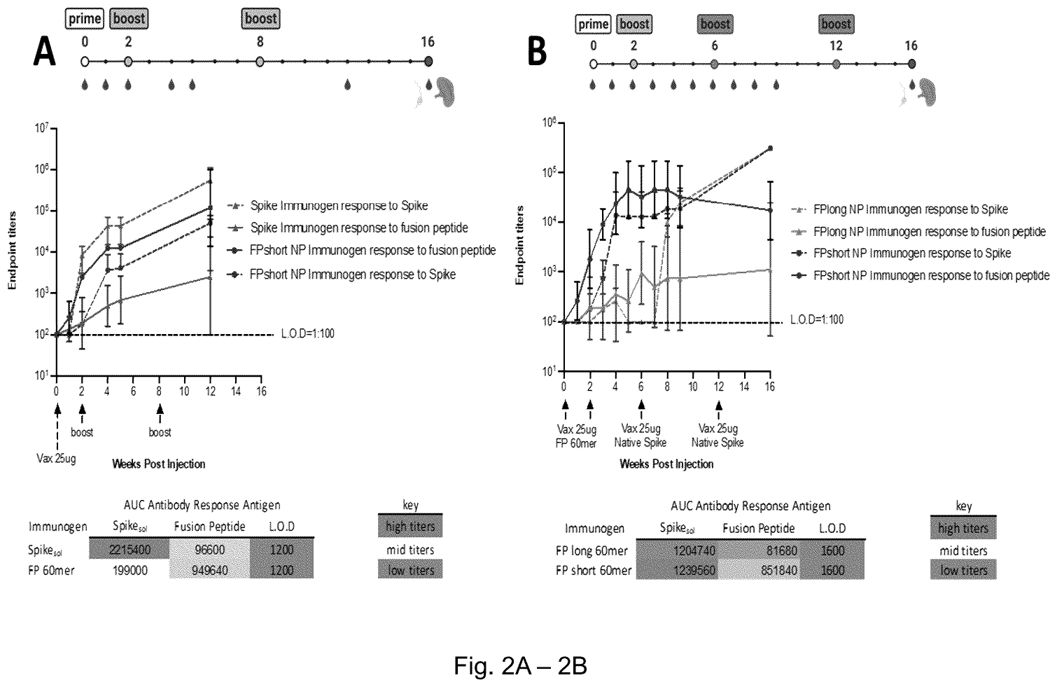

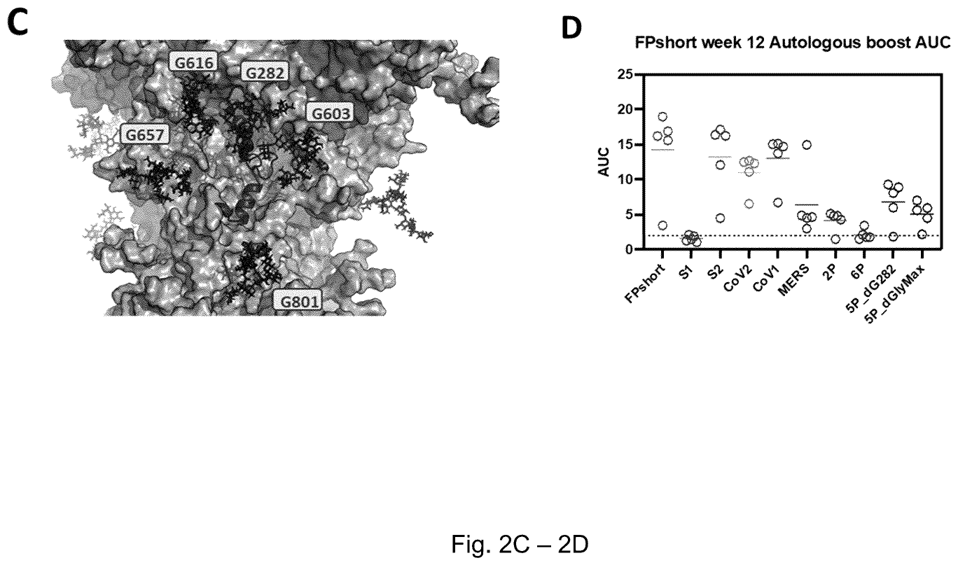

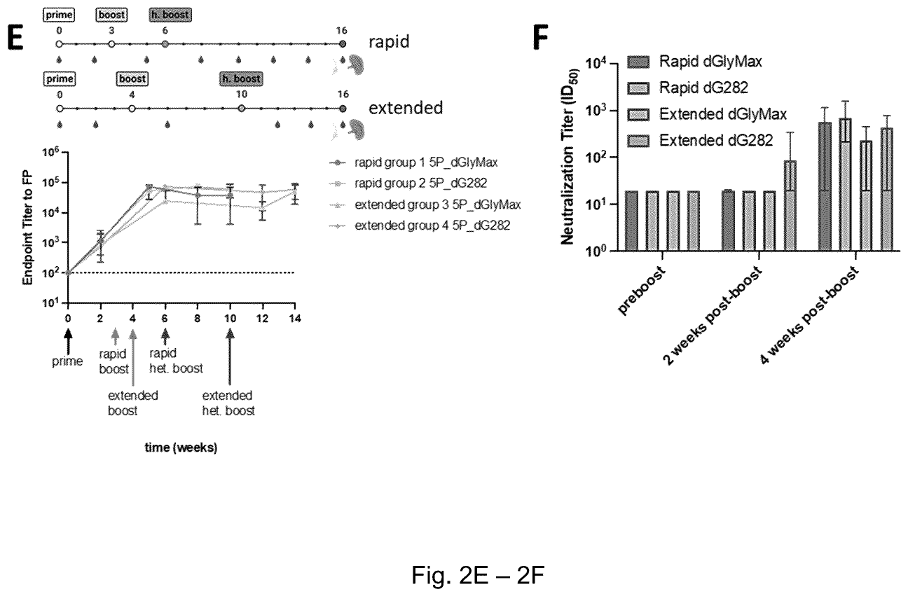

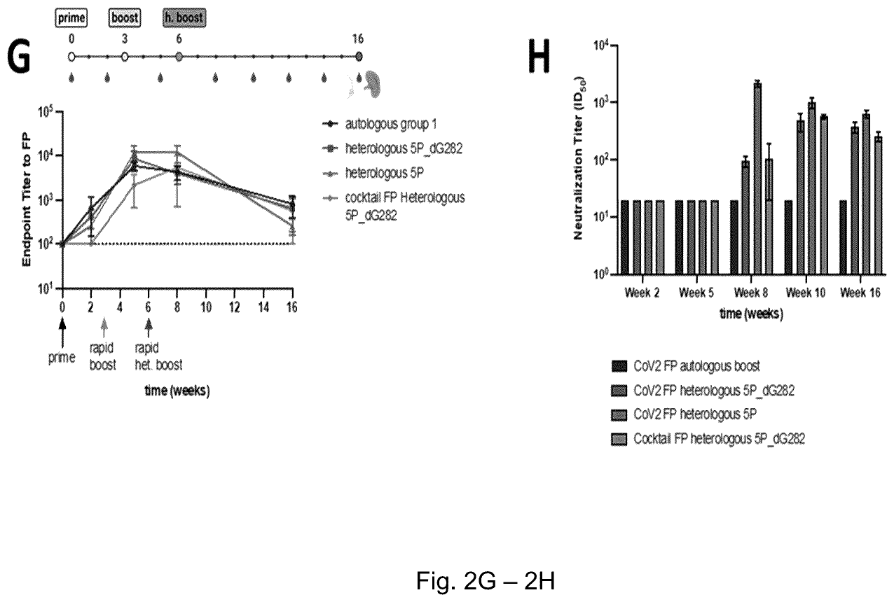

FIG. 2 demonstrates the optimization of boosting strategies for the designed pan-coronavirus in mice. FIG. 2A shows immunogenicity as assessed by endpoint titer following immunization (3 times) with full length spike or fusion peptide nanoparticle.

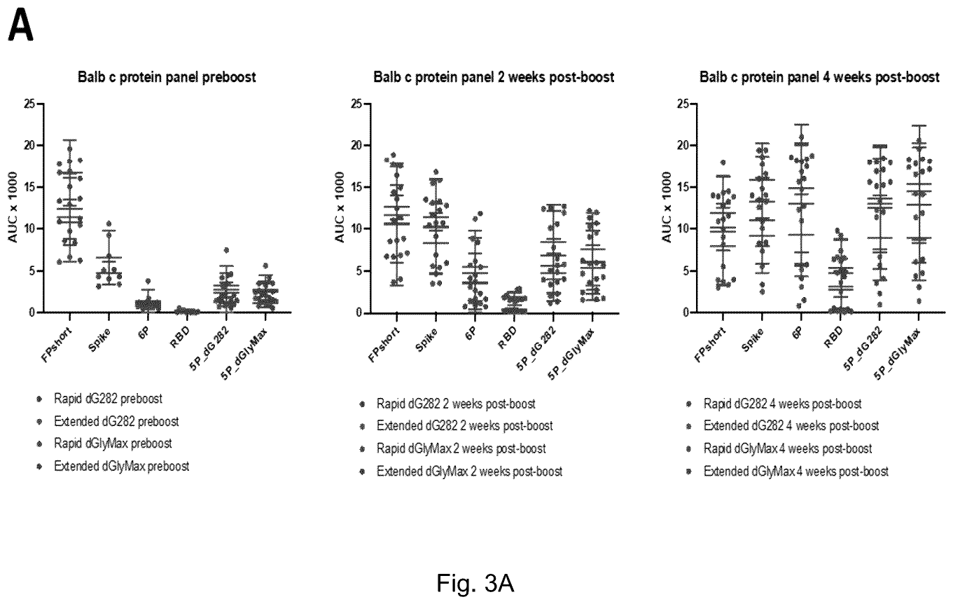

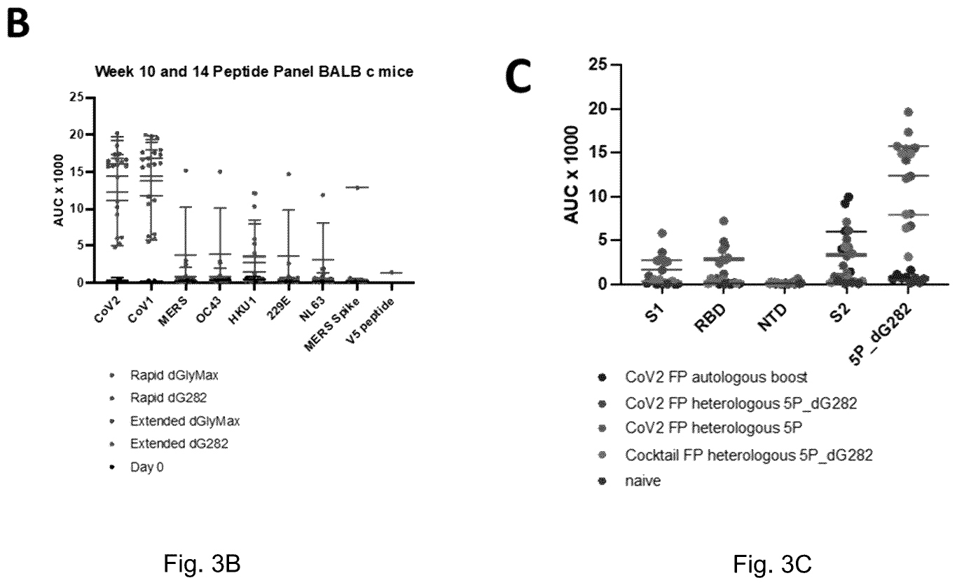

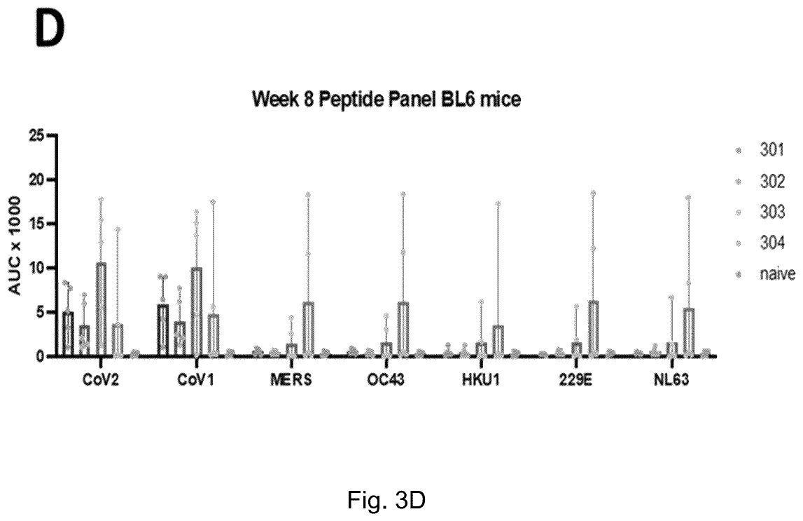

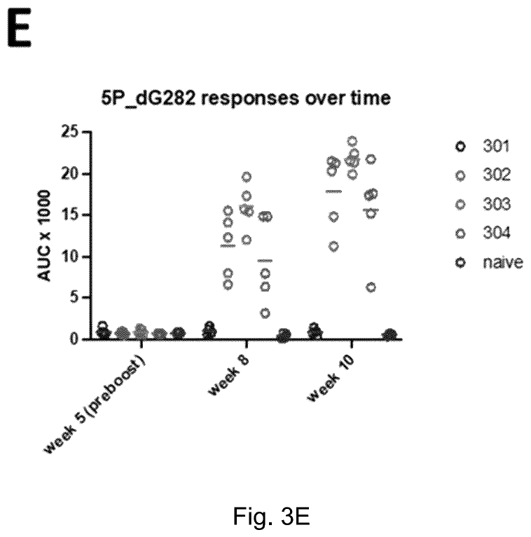

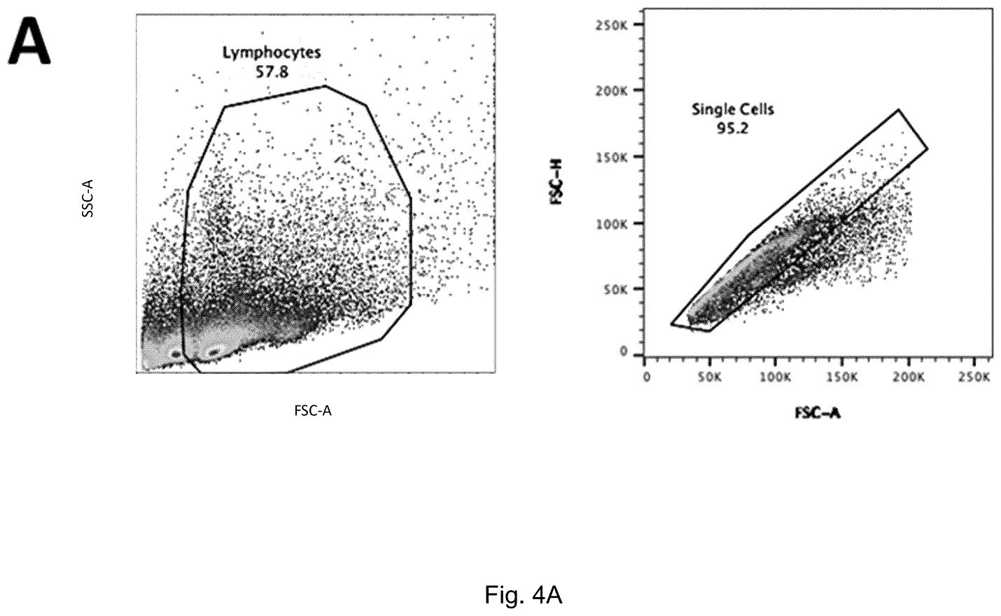

FIG. 2B shows immunogenicity as assessed by endpoint titer following immunizations (4 times) with two different fusion peptide nanoparticles. FIG. 2C depicts a structural model of the fusion peptide epitope with surrounding N-linked glycans. FIG. 2D shows immunogenicity as Area-Under-the-Curve (AUC) analysis as determined by ELISA against SARS-CoV-2 spike protein epitopes after immunization with fusion peptide nanoparticle vaccine. FIG. 2E shows immunogenicity with fusion peptide nanoparticle using a rapid or extended immunization schedule with a heterologous 5P trimer boost. FIG. 2F shows pseudo virus neutralization titers for the experiment in FIG. 2E. FIG. 2G shows immunogenicity after various heterologous boosting antigens. FIG. 2H shows pseudo virus neutralization using sera from experiment in FIG. 2G. FIG. 3 demonstrates cross-reactivity of pan-coronavirus immunogen-immunized mice to various human coronavirus fusion peptides. FIG. 3A shows immunogenicity as Area-Under-the-Curve (AUC) analysis as determined by ELISA against SARS-CoV-2 spike protein epitopes after immunization with fusion peptide nanoparticle vaccine and heterologous boosting. FIG. 3B shows binding to various human coronavirus fusion peptides demonstrating cross-reactivity of the sera. FIG. 3C shows immunogenicity against various epitopes in Black6 mice. FIG. 3D shows immunogenicity as Area-Under-the-Curve (AUC) analysis as determined by ELISA against SARS-CoV-2 spike protein epitopes after immunization with fusion peptide nanoparticle vaccine and heterologous boosting in Black 6 mice. FIG. 3E shows responses to 5P dG282 spike trimer over time.

### Example 2: Pan-Coronavirus Antibodies

The present Example demonstrates the generation and testing of broadly reactive anti-FP peptide monoclonal antibodies. Notably, vaccine-induced monoclonal antibody, C2691, recapitulates serological findings of binding to both beta- and alpha-coronaviruses. Herein the structural basis for this broad recognition to the fusion peptide is defined. C2691 exhibits broad Fc-mediated effector functions and inhibits spike-based cell-to-cell fusion.

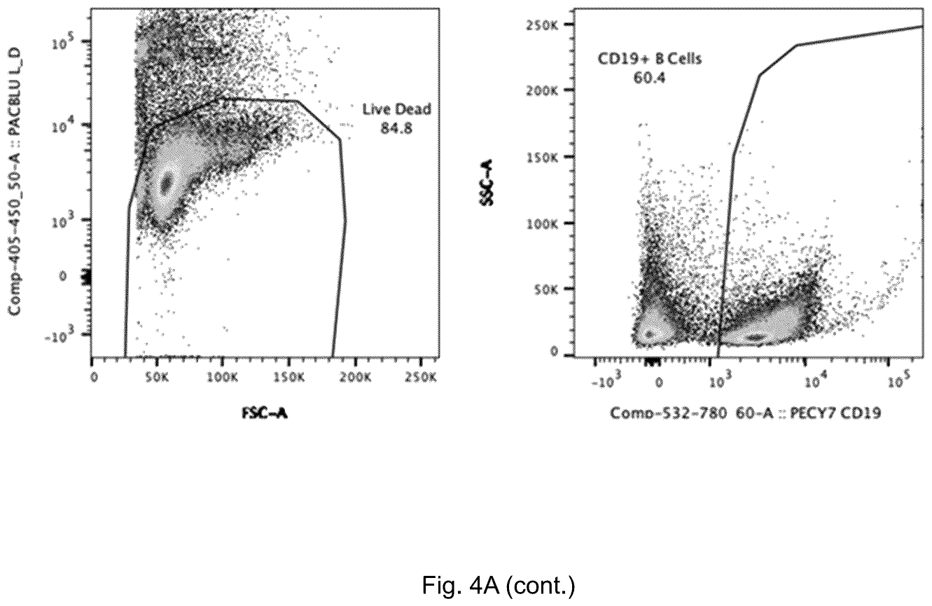

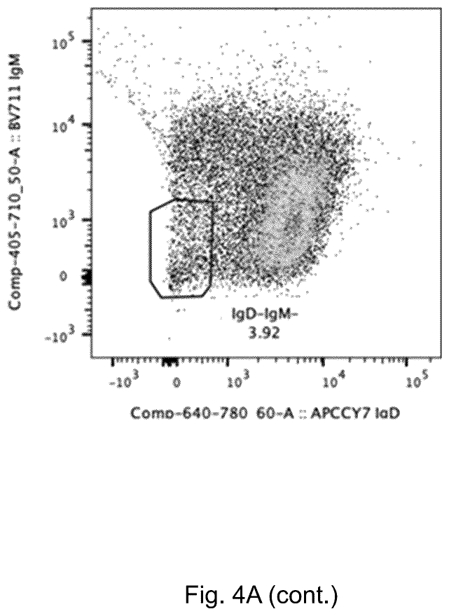

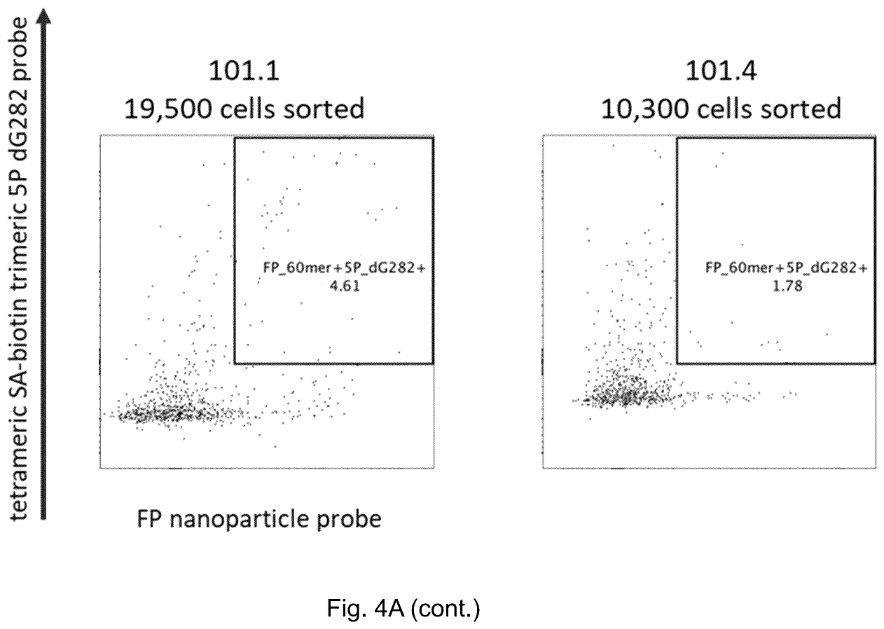

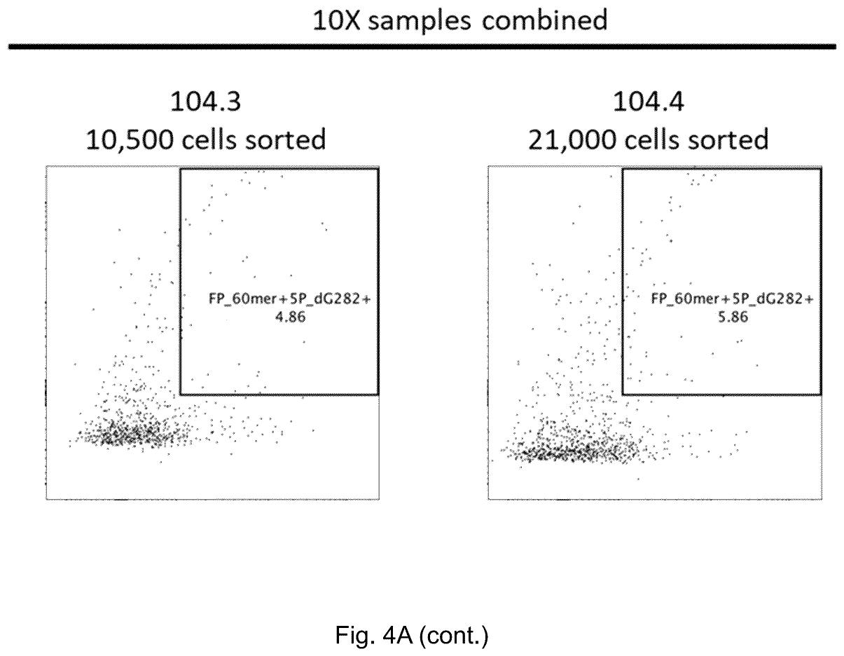

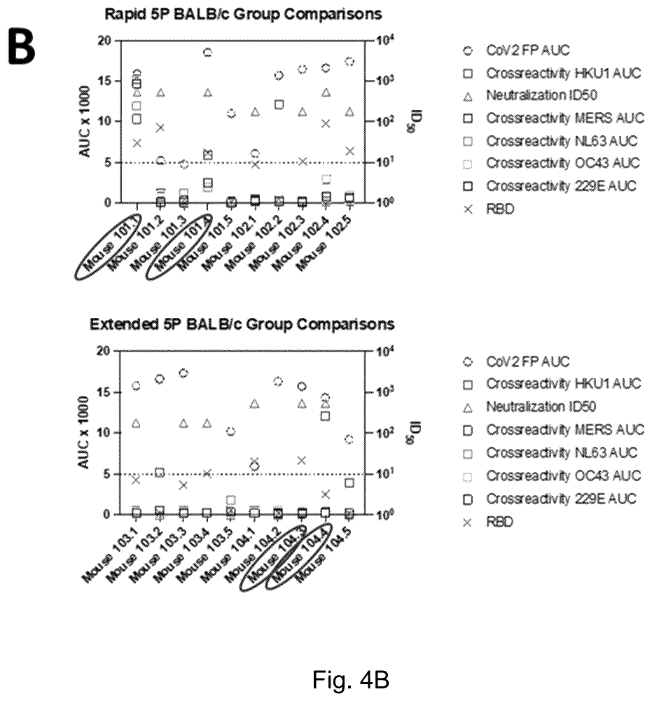

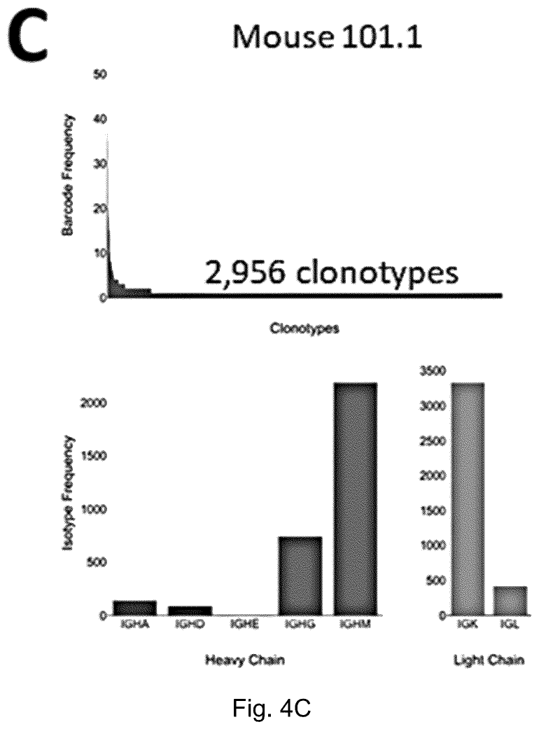

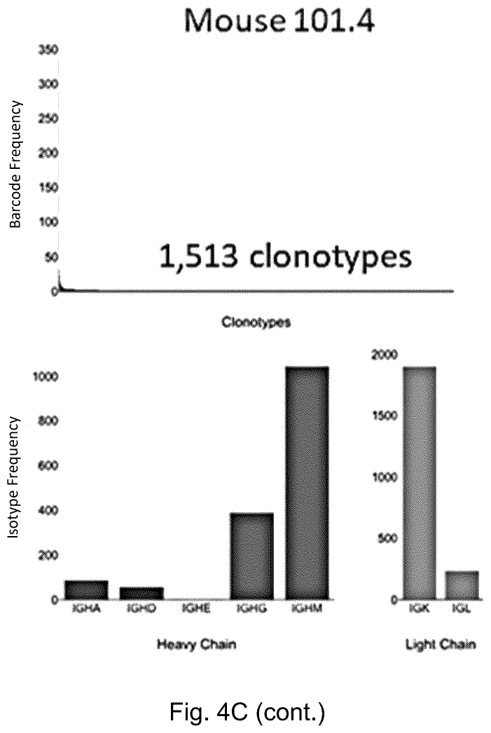

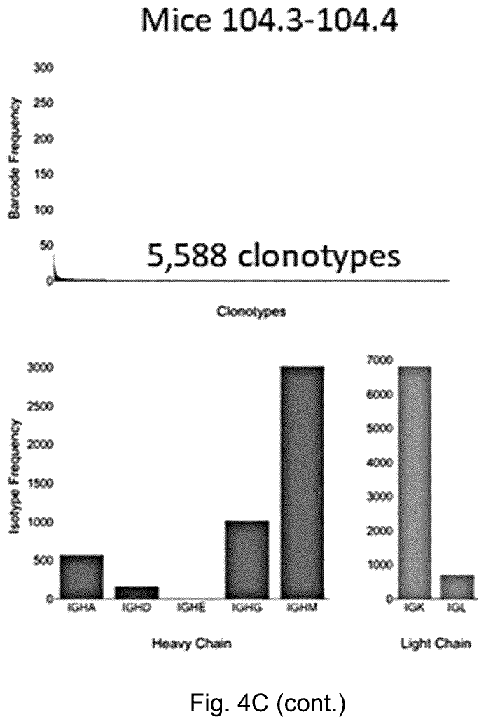

FIG. 4 demonstrates the production of monoclonal antibodies from mice immunize with the pan-coronavirus immunogen as described in Example 1.

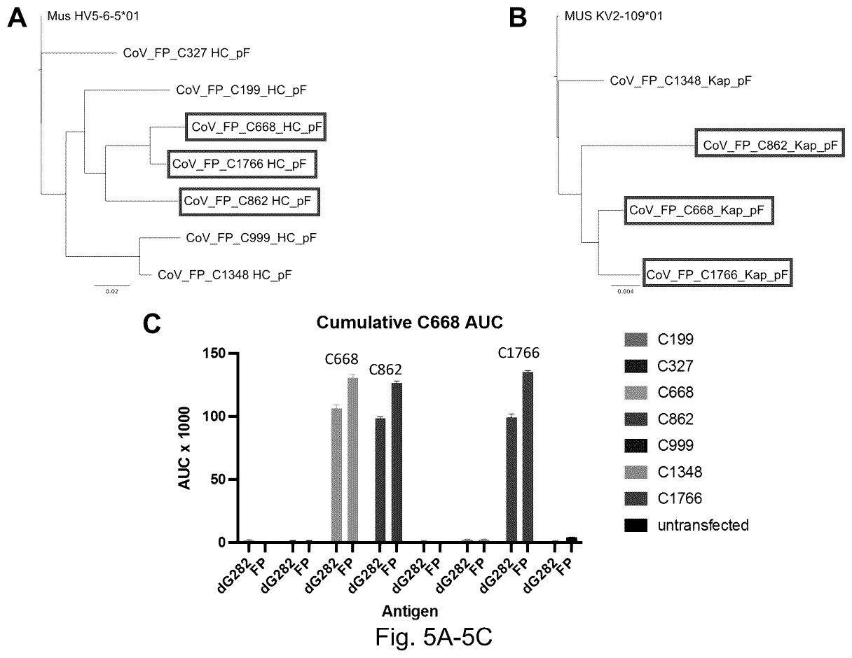

FIG. 5 shows the generation of lineage HV5-6-5/KV2-109 antibodies. A schematic comparison of the CDR3 loops from C668, C862 and C1766 antibody heavy and light chains to Mus HV5-6-5 and Mus KV2-109 demonstrated that while there is little variability of the heavy chain CDR3 loops and no variability of the light chain CDR3 loops, there are moderate somatic hypermutations throughout the remaining variable regions. Antibody C668, C862 and C1766 IgG have 91.8%, 92.8% and 93.8% pairwise identity respectively to the AA HV. Antibody C668, C862 and C1766 IgG have 97.9%, 95.8% and 97.9% pairwise identity respectively to the AA Kap.

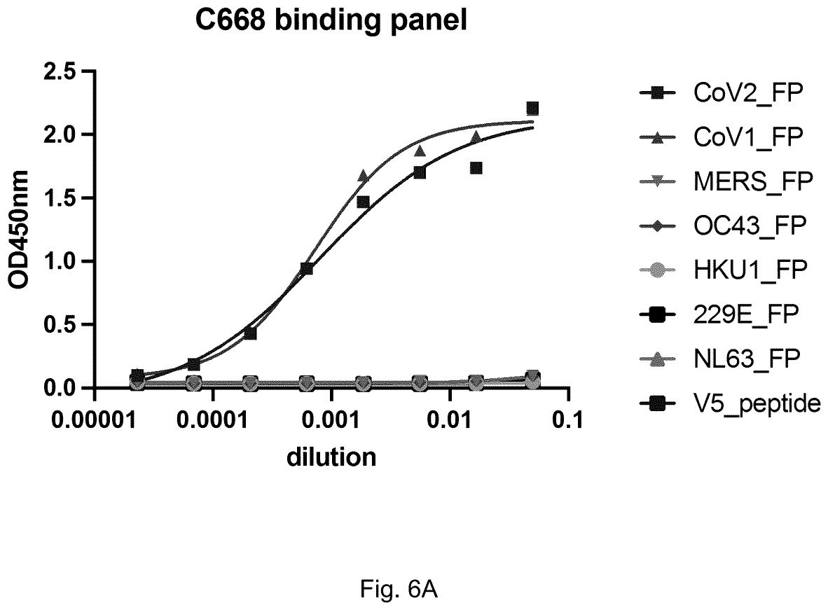

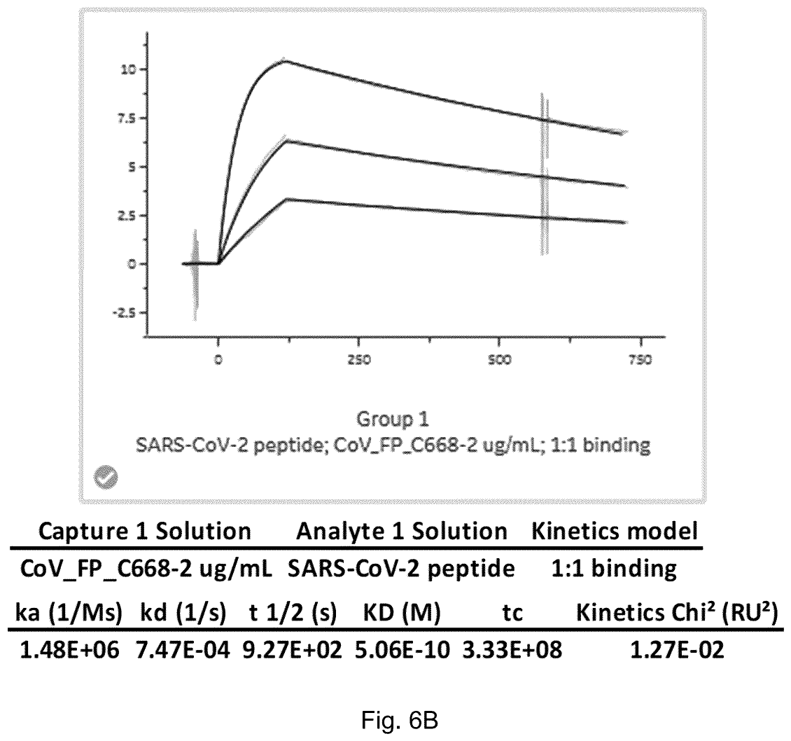

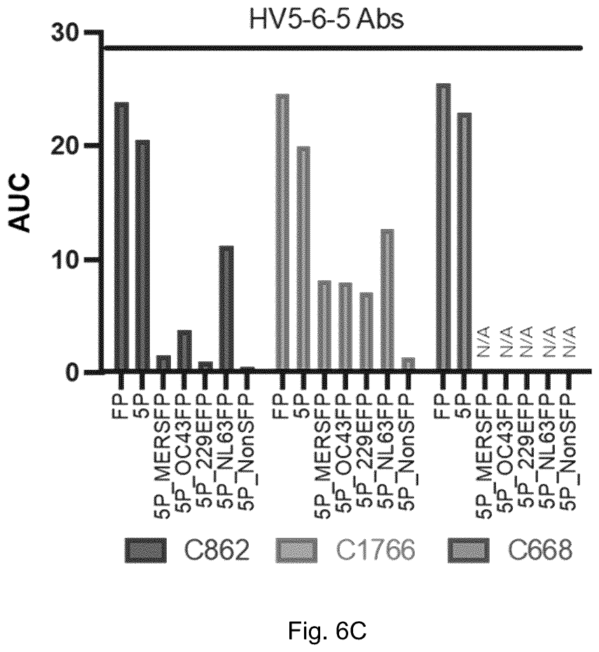

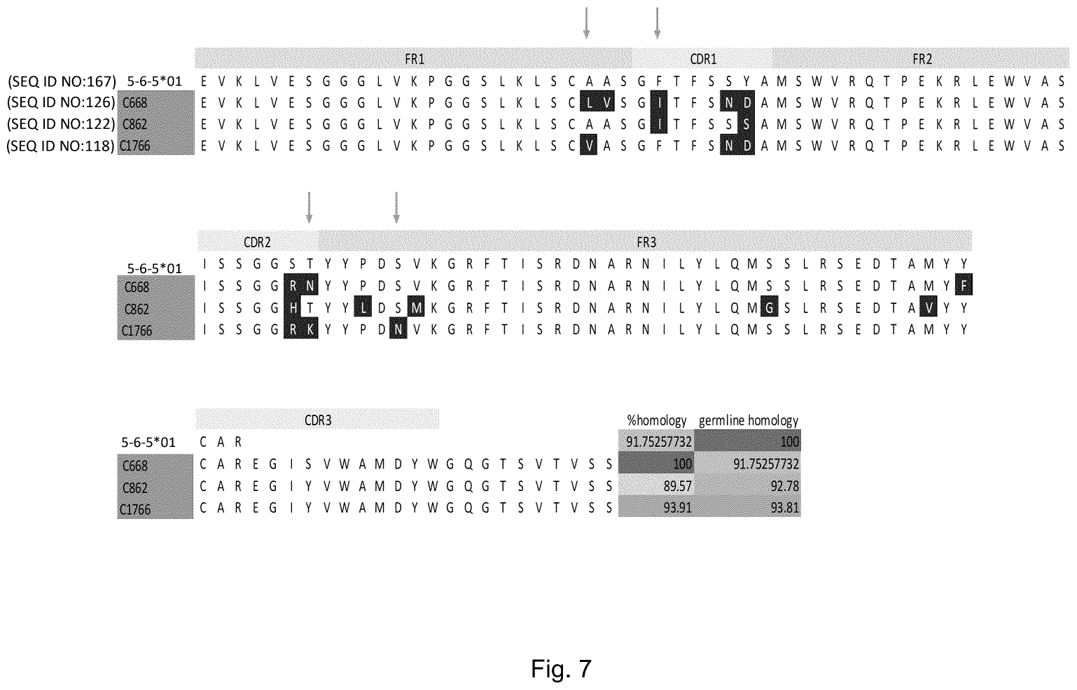

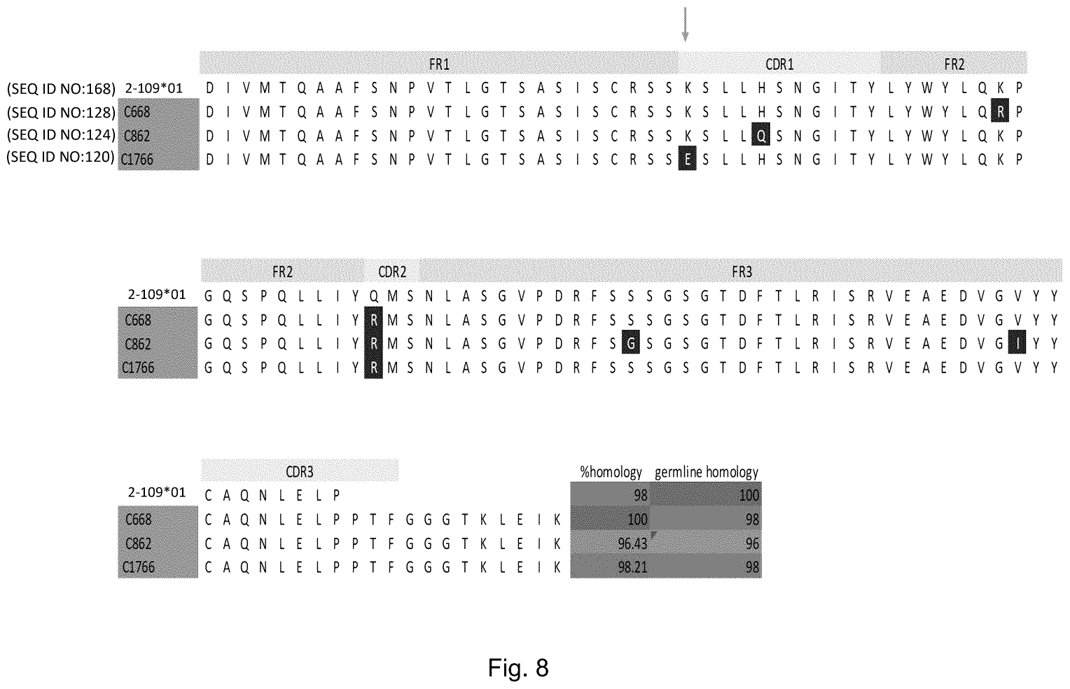

FIG. 6 demonstrates cross-reactivity and high affinity binding of the lineage HV5-6-5/KV2-109 antibodies to various human coronavirus fusion peptides, but not a scrambled fusion peptide sequence. FIGS. 7 and 8 depict schematic comparisons of the C668, C862 and C1766 antibody heavy chains to Mus HV5-6-5 and antibody light chains to Mus KV2-109, respectively.

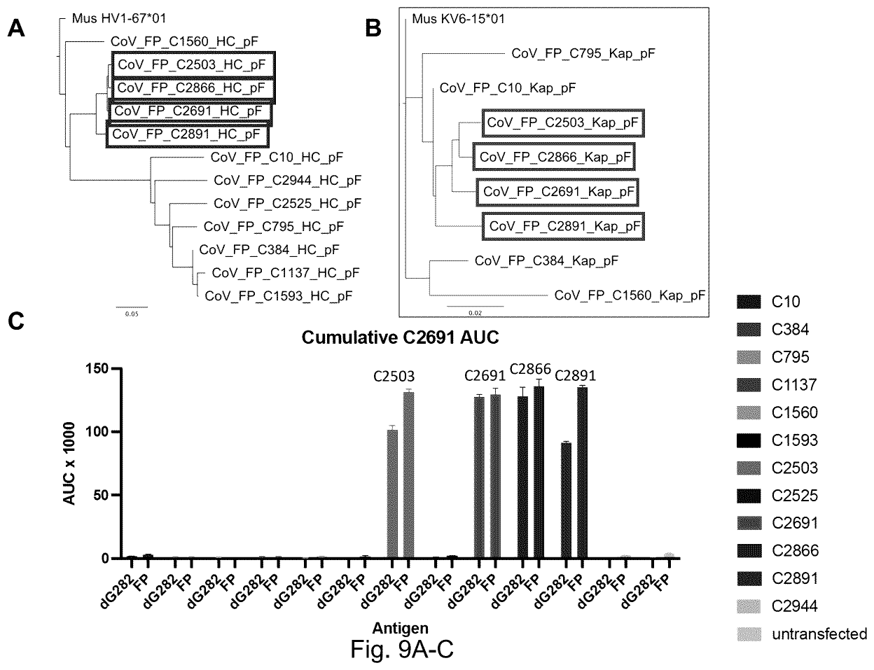

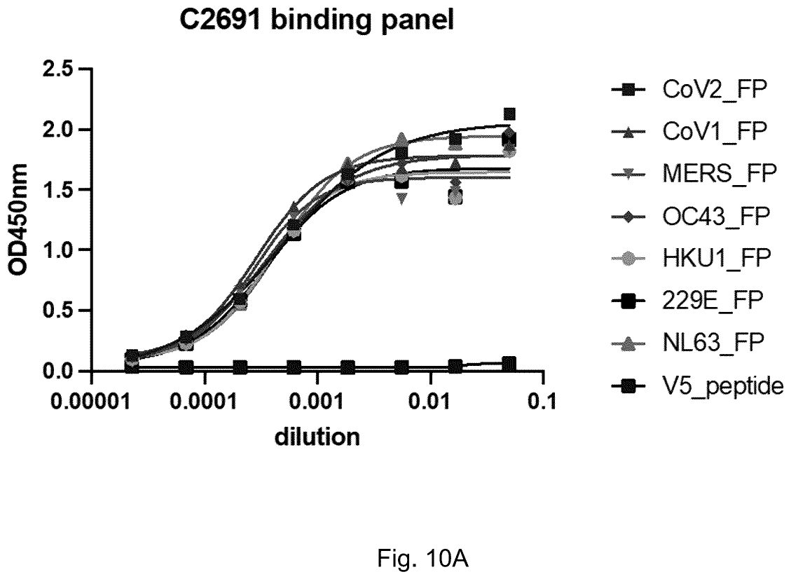

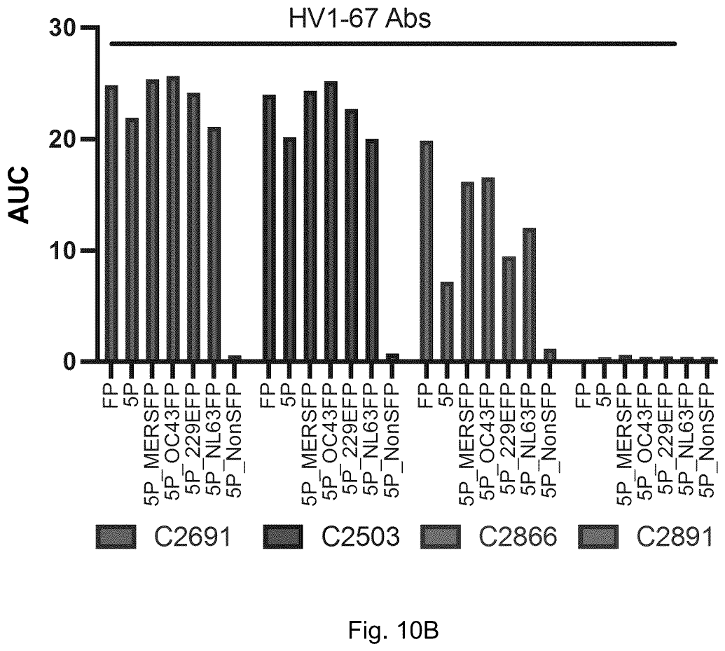

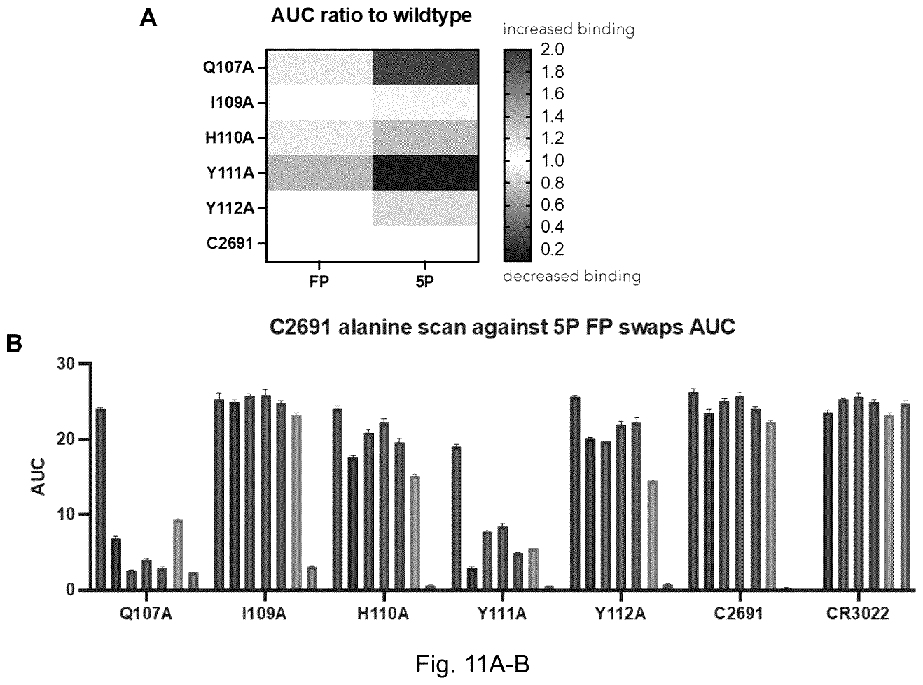

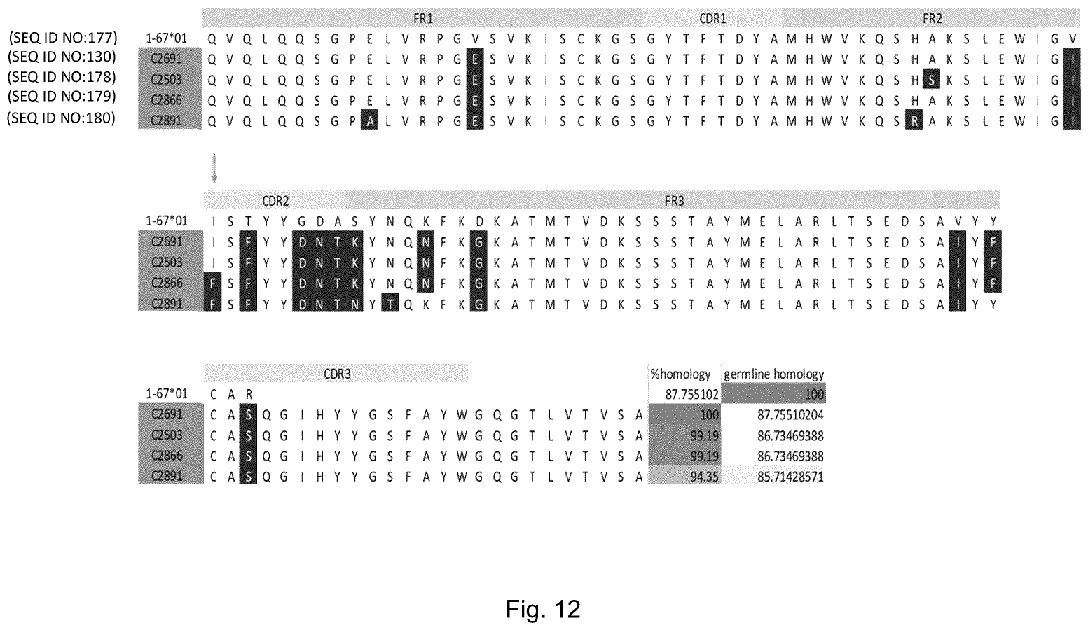

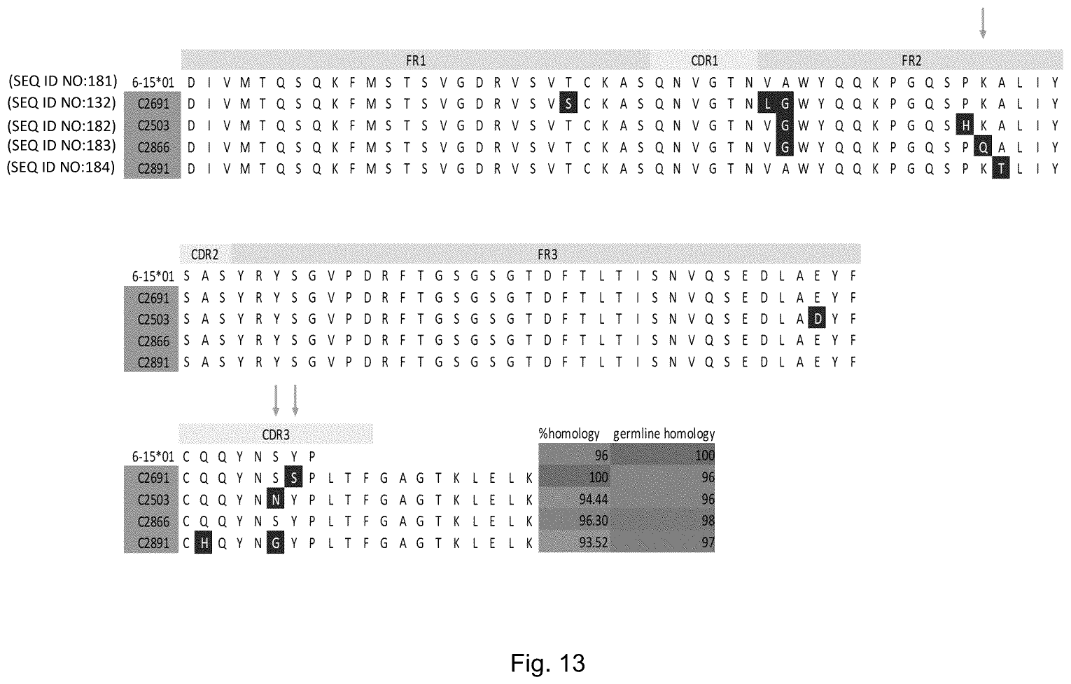

FIG. 9 demonstrates the generation of lineage HV1-67/KV6-15 antibodies. While there is no variability of the heavy chain CDR3 loops and some variability of the light chain CDR3 loops, there are moderate somatic hypermutations throughout the remaining variable regions. A comparison of the CDR3 loops from the C2691, C2503, C2866 and C2891 antibody heavy and light chains to Mus HV1-67 and Mus KV6-15 revealed that C2691, C2503, C2866 and C2891 have 87.8%, 86.7%, 86.7% and 85.7% pairwise identity respectively to AA HV, and C2691, C2503, C2866 and C2891 have 95.8%, 95.8%, 97.9% and 96.8% pairwise identity respectively to AA Kap. FIG. 10 demonstrates cross-reactivity of the lineage HV1-67/KV6-15 antibodies to various human coronavirus fusion peptides, but not a scrambled fusion peptide sequence. FIG. 11 shows lineage HV1-67/KV6-15 HCDR3 loop interactions. Notably, a reduction in affinity is observed for antibodies with alanine mutations at positions 107 and 111 against all human coronavirus conformational epitopes. FIGS. 12 and 13 depict schematic comparisons of the C2691, C2503, C2866 and C2891 antibody heavy chains to Mus HV1-67 and antibody light chains to Mus KV6-15, respectively.

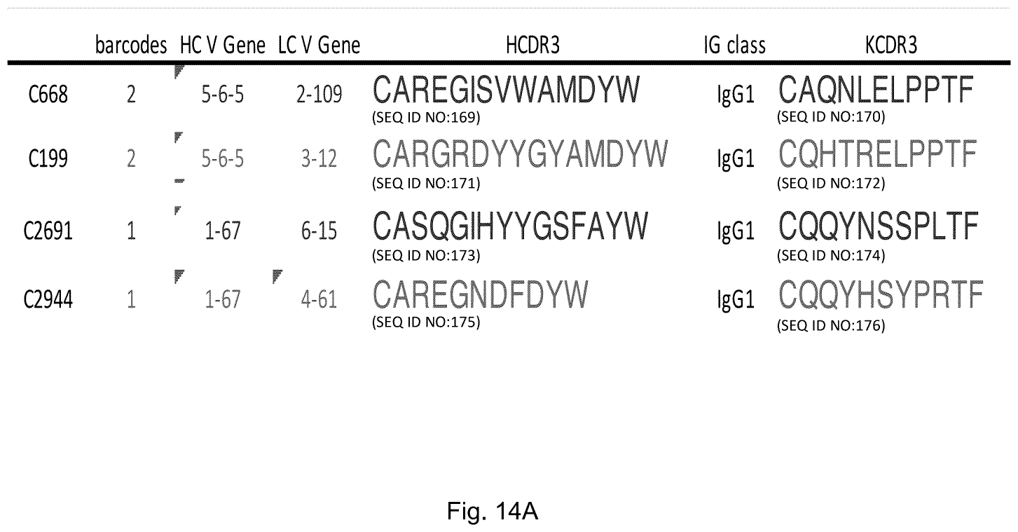

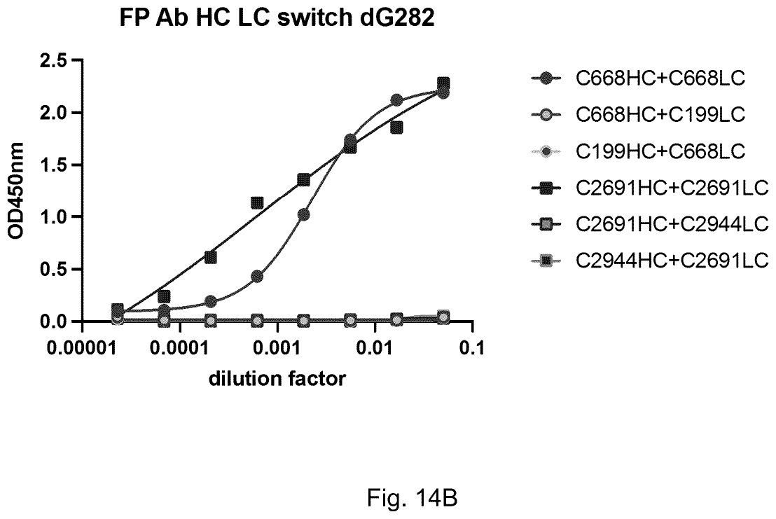

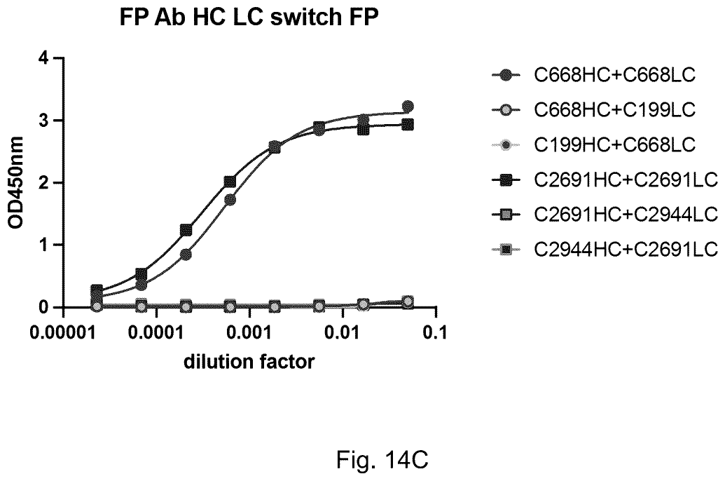

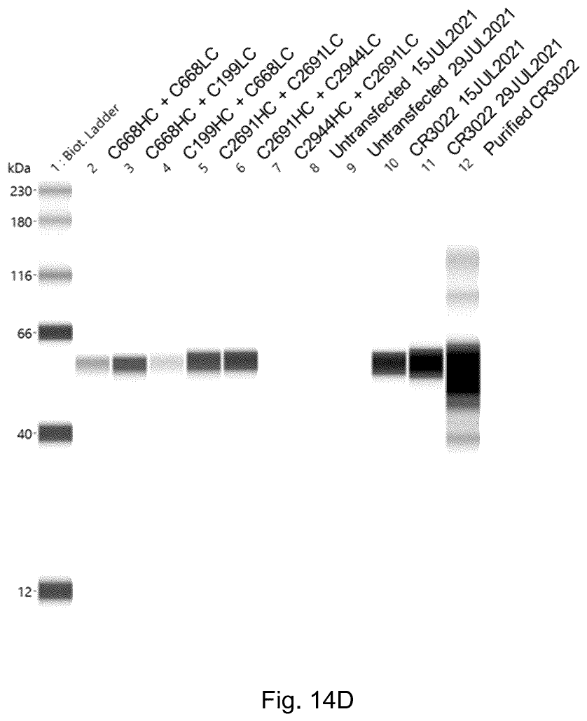

FIG. 14 shows that both the heavy chain and the light chain for lineage HV5-6-5/KV2-109 antibody C668 and lineage HV1-67/KV6-15 antibody C2691 are important for binding to human coronavirus fusion peptides. FIG. 14A shows the genetic features of the antibodies. FIG. 14B shows binding of heavy/light chain swapped antibodies to 5P spike. FIG. 14C shows binding of heavy/light chain swapped antibodies to fusion peptide. FIG. 14D shows expression of heavy/light chain swapped antibodies.

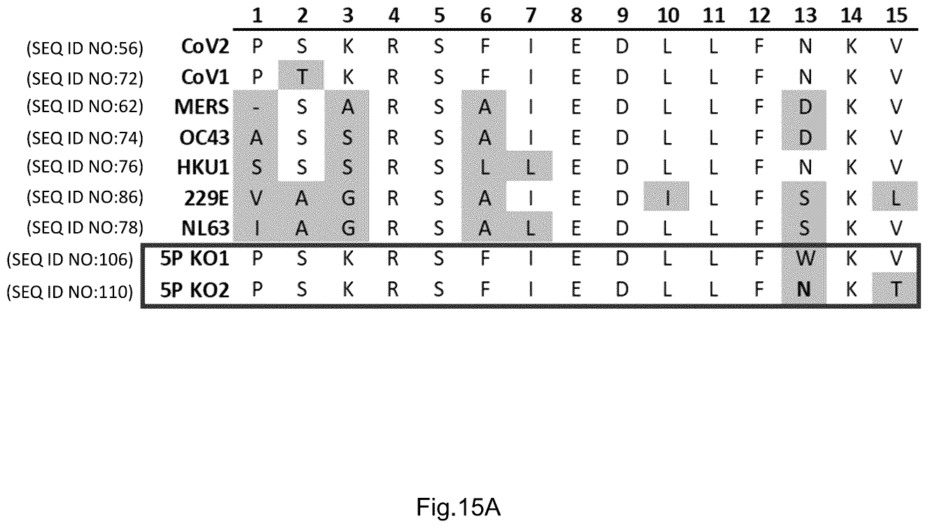

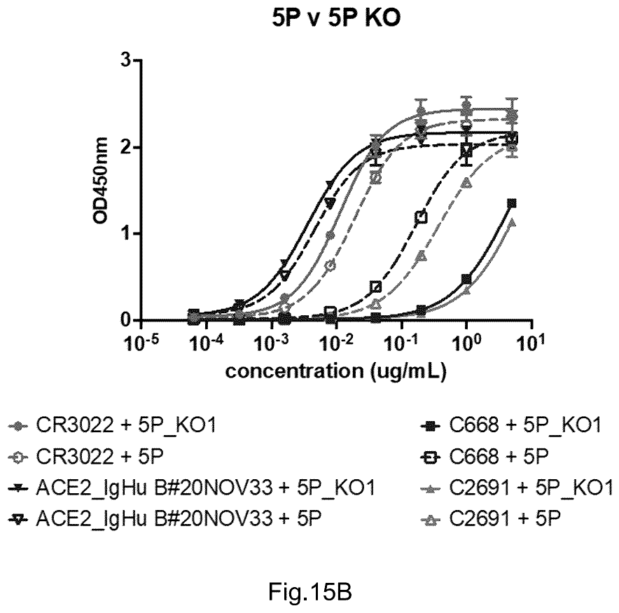

FIG. 15 demonstrates that position 13 on the CoV2 fusion peptide can knock down antibody binding. FIG. 15A shows sequences of fusion peptide from diverse coronaviruses and the present 5P KO1 and 5P KO2 proteins are designed to have low affinity for fusion peptide antibodies. FIG. 15B shows reduced binding of C668 and C2691 for the 5P KO1 construct.

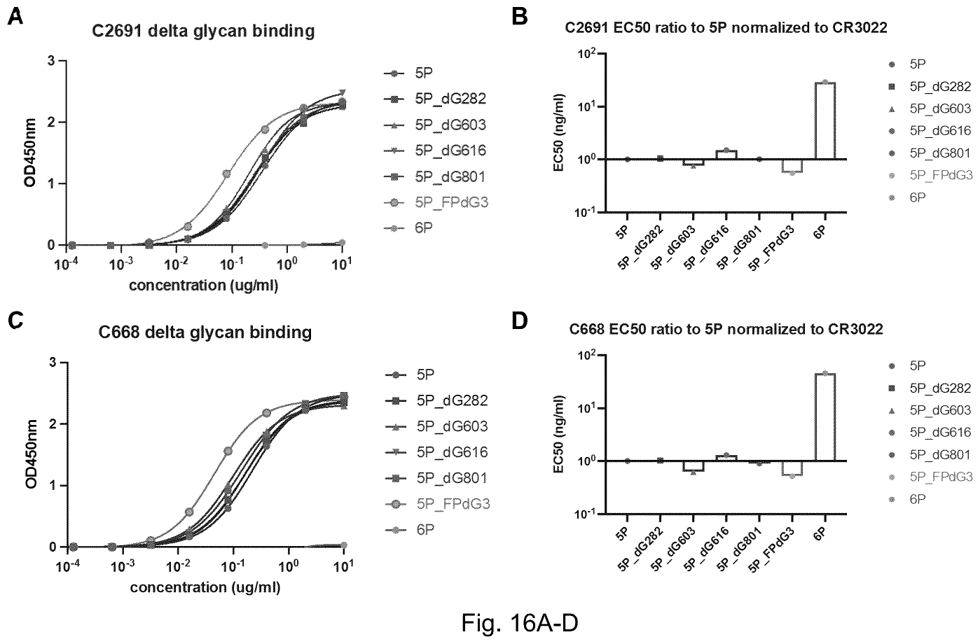

Finally, FIG. 16 demonstrates that multiple glycan deletions aid in antibody binding to human coronavirus fusion peptides. FIG. 16A shows binding of C2691 to 5P spike proteins with glycans removed near-by the fusion peptide. FIG. 16B shows data in Panel A as EC50. FIG. 16C shows binding of C668 to 5P spike proteins with glycans removed near-by the fusion peptide. FIG. 16D depicts data in FIG. 16C as EC50.

### Example 3—Sequences

| | | Sequence |
|---|---|---|
| SEQ ID NO: | Description | Type |
| 1 | CoV2_FP_L9GT60_pVax | DNA |
| 2 | CoV2_FP_L9GT60_pVax | PRT |
| 3 | CoV2_FP12_L9GT60_pVax | DNA |
| 4 | CoV2_FP12_L9GT60_pVax | PRT |
| 5 | 229E_FP_L9GT60_pVax | DNA |
| 6 | 229E_FP_L9GT60_pVax | PRT |
| 7 | MERS_FP_L9GT60_pVax | DNA |
| 8 | MERS_FP_L9GT60_pVax | PRT |
| 9 | NL63_FP_L9GT60_pVax | DNA |
| 10 | NL63_FP_L9GT60_pVax | PRT |
| 11 | CoV2_FPX2_L9GT60_pVax | DNA |
| 12 | CoV2_FPX2_L9GT60_pVax | PRT |
| 13 | CoV2_FP_CA24mer_pVax | DNA |
| 14 | CoV2_FP_CA24mer_pVax | PRT |
| 15 | CoV1_FP_CA24mer_pVax | DNA |
| 16 | CoV1_FP_CA24mer_pVax | PRT |
| 17 | MERS_FP_CA24mer_pVax | DNA |
| 18 | MERS_FP_CA24mer_pVax | PRT |
| 19 | OC43_FP_CA24mer_pVax | DNA |
| 20 | OC43_FP_CA24mer_pVax | PRT |
| 21 | HKU1_FP_CA24mer_pVax | DNA |
| 22 | HKU1_FP_CA24mer_pVax | PRT |
| 23 | 229E_FP_CA24mer_pVax | DNA |
| 24 | 229E_FP_CA24mer_pVax | PRT |
| 25 | NL63_FP_CA24mer_pVax | DNA |
| 26 | NL63_FP_CA24mer_pVax | PRT |
| 27 | CoV2_5P_pVax | DNA |
| 28 | CoV2_5P_pVax | PRT |
| 29 | CoV2_5P_dG282_pVax | DNA |
| 30 | CoV2_5P_dG282_pVax | PRT |
| 31 | CoV2_5P_FPdGlyMax_pVax | DNA |
| 32 | CoV2_5P_FPdGlyMax_pVax | PRT |
| 33 | CoV2_5P_229EFP_pVax | DNA |
| 34 | CoV2_5P_229EFP_pVax | PRT |
| 35 | CoV2_5P_HKU1FP_pVax | DNA |
| 36 | CoV2_5P_HKU1FP_pVax | PRT |
| 37 | CoV2_5P_MERSFP_pVax | DNA |
| 38 | CoV2_5P_MERSFP_pVax | PRT |
| 39 | CoV2_5P_NL63FP_pVax | DNA |
| 40 | CoV2_5P_NL63FP_pVax | PRT |
| 41 | CoV2_5P_OC43FP_pVax | DNA |
| 42 | CoV2_5P_OC43FP_pVax | PRT |
| 43 | CoV2_5P_FPKO1_pVax | DNA |
| 44 | CoV2_5P_FPKO1_pVax | PRT |
| 45 | CoV2_5P_FPKO2_pVax | DNA |
| 46 | CoV2_5P_FPKO2_pVax | PRT |
| 47 | CoV2_5P_NonSFP_pVax | DNA |
| 48 | CoV2_5P_NonSFP_pVax | PRT |
| 49 | IgE Leader Sequence | DNA |
| 50 | IgE Leader Sequence | PRT |
| 51 | GGS Linker | DNA |
| 52 | GGS Linker | PRT |
| 53 | GT8-LS-60mer | DNA |
| 54 | GT8-LS-60mer | PRT |
| 55 | CoV2 Fusion Peptide Short | DNA |
| 56 | CoV2 Fusion Peptide Short | PRT |
| 57 | CoV2 Fusion Peptide Long | DNA |
| 58 | CoV2 Fusion Peptide Long | PRT |
| 59 | 229E Fusion Peptide Short | DNA |
| 60 | 229E Fusion Peptide Short | PRT |
| 61 | MERS Fusion Peptide Short | DNA |
| 62 | MERS Fusion Peptide Short | PRT |
| 63 | NL63 Fusion Peptide Short | DNA |
| 64 | NL63 Fusion Peptide Short | PRT |
| 65 | CoV2 Fusion Peptide Short x2 | DNA |
| 66 | CoV2 Fusion Peptide Short x2 | PRT |
| 67 | CA09-FR-24mer | DNA |
| 68 | CA09-FR-24mer | PRT |
| 69 | CoV2 Fusion Peptide Short v2 | DNA |
| 70 | CoV2 Fusion Peptide Short v2 | PRT |
| 71 | CoV1 Fusion Peptide Short | DNA |
| 72 | CoV1 Fusion Peptide Short | PRT |
| 73 | OC43 Fusion Peptide Short | DNA |
| 74 | OC43 Fusion Peptide Short | PRT |
| 75 | HKU1 Fusion Peptide Short | DNA |
| 76 | HKU1 Fusion Peptide Short | PRT |
| 77 | NL63 Fusion Peptide Short v2 | DNA |
| 78 | NL63 Fusion Peptide Short v2 | PRT |
| 79 | CoV2_5P | DNA |
| 80 | CoV2_5P | PRT |
| 81 | CoV2_5P_dG282 | DNA |
| 82 | CoV2_5P_dG282 | PRT |
| 83 | CoV2_5P_FPdGlyMax | DNA |
| 84 | CoV2_5P_FPdGlyMax | PRT |
| 85 | 229EFP v2 | DNA |
| 86 | 229EFP | PRT |
| 87 | CoV2_5P_229EFP | DNA |
| 88 | CoV2_5P_229EFP | PRT |
| 89 | HKU1FP v2 | DNA |
| 90 | HKU1FP | PRT |
| 91 | CoV2_5P_HKU1FP | DNA |
| 92 | CoV2_5P_HKU1FP | PRT |
| 93 | MERSFP v2 | DNA |
| 94 | MERSFP v2 | PRT |
| 95 | CoV2_5P_MERSFP | DNA |
| 96 | CoV2_5P_MERSFP | PRT |
| 97 | NL63FP | DNA |
| 98 | NL63FP | PRT |
| 99 | CoV2_5P_NL63FP | DNA |
| 100 | CoV2_5P_NL63FP | PRT |
| 101 | OC43FP | DNA |
| 102 | OC43FP | PRT |
| 103 | CoV2_5P_OC43FP | DNA |
| 104 | CoV2_5P_OC43FP | PRT |
| 105 | FPKO1 | DNA |
| 106 | FPKO1 | PRT |
| 107 | CoV2_5P_FPKO1 | DNA |
| 108 | CoV2_5P_FPKO1 | PRT |
| 109 | FPKO2 | DNA |
| 110 | FPKO2 | PRT |
| 111 | CoV2_5P_FPKO2 | DNA |
| 112 | CoV2_5P_FPKO2 | PRT |
| 113 | NonSFP | DNA |
| 114 | NonSFP | PRT |
| 115 | CoV2_5P_NonSFP | DNA |
| 116 | CoV2_5P_NonSFP | PRT |
| 117 | CoV_FP_C1766_HC_pF | DNA |
| 118 | CoV_FP_C1766_HC_pF | PRT |
| 119 | CoV_FP_C1766_kap_pF | PRT |
| 120 | CoV_FP_C1766_kap_pF | DNA |
| 121 | CoV_FP_C862_HC_pF | PRT |
| 122 | CoV_FP_C862_HC_pF | DNA |
| 123 | CoV_FP_C862_kap_pF | PRT |
| 124 | CoV_FP_C862_kap_pF | DNA |
| 125 | CoV_FP_C668_HC_pF | PRT |
| 126 | CoV_FP_C668_HC_pF | PRT |
| 127 | CoV_FP_C668_Kap_pF | DNA |
| 128 | CoV_FP_C668_Kap_pF | PRT |
| 129 | CoV_FP_C2691_HC_pF | DNA |
| 130 | CoV_FP_C2691_HC_pF | PRT |
| 131 | CoV_FP_C2691_Kap_pF | DNA |
| 132 | CoV_FP_C2691_Kap_pF | PRT |
| 133 | C2691_IgG1_ZM1_Hole_myc_pVax | PRT |
| 134 | S2P6_IgG1_ZM1_Knob_V5_pVax | PRT |
| 135 | B6_IgG1_ZM1_Knob_V5_pVax | PRT |
| 136 | 28D9_IgG1_ZM1_Knob_V5_pVax | PRT |
| 137 | C524_IgG1_ZM1_Knob_V5_pVax | PRT |
| 138 | C251_IgG1_ZM1_Knob_V5_pVax | PRT |
| 139 | C217_IgG1_ZM1_Knob_V5_pVax | PRT |
| 140 | C2691_Kap_pVax | PRT |
| 141 | S2P6_Kap_pVax | PRT |
| 142 | B6_Kap_pVax | PRT |
| 143 | 28D9_Kap_pVax | PRT |
| 144 | C524_Kap_pVax | PRT |
| 145 | C217_Kap_pVax | PRT |
| 146 | C251_Lam_pVax | PRT |
| 147 | C524_HC_pFuse | PRT |
| 148 | C251_HC_pFuse | PRT |
| 149 | C217_HC_pFuse | PRT |
| 150 | C524_Kap_pFuse | PRT |
| 151 | C217_Kap_pFuse | PRT |
| 152 | C251_Lam_pFuse | PRT |
| 153 | Kappa | PRT |
| 154 | Lambda | PRT |
| 156 | IgKappa leader sequence | PRT |
| 157 | myc tag | PRT |
| 158 | V5 tag | PRT |

While not being bound by scientific theory, the present Examples demonstrate the generation of novel pan-coronavirus (alpha- and beta-) vaccines and antibodies for the treatment and prevention of coronavirus infection.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A pan-coronavirus immunogenic composition comprising one or more selected from the group consisting of:
a) one or more coronavirus antigenic polypeptide;
b) one or more nucleic acid molecule encoding one or more coronavirus antigenic polypeptide;
c) one or more coronavirus monoclonal antibody; and
d) one or more nucleic acid molecule encoding one or more coronavirus monoclonal antibody.

2. The pan-coronavirus immunogenic composition of claim 1, wherein said coronavirus antigenic polypeptide comprises a coronavirus fusion peptide (FP).

3. The pan-coronavirus immunogenic composition of claim 2, wherein said coronavirus FP comprises one or more selected from the group consisting of:
a) one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114;
b) a polypeptide comprising an amino acid sequence at least 90% identical to one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114;
c) a polypeptide comprising an amino acid sequence at least 70% of the length of one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114; and
d) a polypeptide comprising an amino acid sequence at least 90% identical to and at least 70% the length of one or more coronavirus FP selected from the group consisting of: SEQ ID NO: 56, SEQ ID NO: 58, SEQ ID NO: 60, SEQ ID NO: 62, SEQ ID NO: 64, SEQ ID NO: 66, SEQ ID NO: 72, SEQ ID NO: 74, SEQ ID NO: 76, SEQ ID NO: 94, SEQ ID NO: 106, SEQ ID NO: 110, and SEQ ID NO: 114.

4. The pan-coronavirus immunogenic composition of claim 2, wherein said coronavirus antigenic polypeptide comprises a prefusion stabilized spike protein from one or more coronavirus.

5. The pan-coronavirus immunogenic composition of claim 4, wherein said coronavirus antigenic polypeptide comprises one or more amino acid sequence selected from the group consisting of: SEQ ID NO: 80, SEQ ID NO: 82, SEQ ID NO: 84, SEQ ID NO: 88, SEQ ID NO: 92, SEQ ID NO: 96, SEQ ID NO: 100, SEQ ID NO: 104, SEQ ID NO: 108, SEQ ID NO: 112, SEQ ID NO: 116.

6. The pan-coronavirus immunogenic composition of claim 1, wherein said one or more coronavirus antigenic polypeptide further comprises a nanoparticle scaffold.

7. The pan-coronavirus immunogenic composition of claim 4, wherein said nanoparticle scaffold comprises at least one of: a GT8-LS-60mer nanoparticle scaffold (SEQ ID NO: 54) and a CA09-FR-24mer nanoparticle scaffold (SEQ ID NO: 68).

8. The pan-coronavirus immunogenic composition of claim 1, wherein said nucleic acid molecule encoding one or more coronavirus antigenic polypeptide comprises one or more selected from the group consisting of:
a) one or more nucleotide sequence selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113;
b) a nucleotide sequence at least 90% identical to one or more selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113;
c) a nucleotide sequence at least 70% of the length of one or more selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113; and
d) a nucleotide sequence at least 90% identical to and at least 70% the length of one or more selected from the group consisting of: SEQ ID NO: 55, SEQ ID NO: 57, SEQ ID NO: 59, SEQ ID NO: 61, SEQ ID NO: 63, SEQ ID NO: 65, SEQ ID NO: 69, SEQ ID NO: 71, SEQ ID NO: 73, SEQ ID NO: 75, SEQ ID NO: 77, SEQ ID NO: 85, SEQ ID NO: 89, SEQ ID NO: 93, SEQ ID NO: 97, SEQ ID NO: 101, SEQ ID NO: 105, SEQ ID NO: 109, and SEQ ID NO: 113.

9. The pan-coronavirus immunogenic composition of claim 8, wherein said nucleic acid molecule further comprises a nucleotide sequence encoding a nanoparticle scaffold, wherein said nucleotide sequence is at least one selected from the group consisting of:
SEQ ID NO: 53 and SEQ ID NO: 67.

10. The pan-coronavirus immunogenic composition of claim 8, wherein said nucleic acid molecule comprises a plasmid vector suitable for mammalian expression.

11. The pan-coronavirus immunogenic composition of claim 10, wherein said plasmid vector comprises one or more nucleotide sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47.

12. The pan-coronavirus immunogenic composition of claim 1, wherein said coronavirus monoclonal antibody comprises a heavy chain, and wherein said heavy chain comprises one or more selected from the group consisting of:
a) a heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149;
b) a polypeptide comprising an amino acid sequence at least 90% identical to one or more heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149;
c) a polypeptide comprising an amino acid sequence at least 70% of the length of one or more heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149; and
d) a polypeptide comprising an amino acid sequence at least 90% identical to and at least 70% the length of one or more heavy chain amino acid sequence selected from the group consisting of: SEQ ID NO: 117, SEQ ID NO: 118, SEQ ID NO: 121, SEQ ID NO: 122, SEQ ID NO: 125, SEQ ID NO: 126, SEQ ID NO: 129, SEQ ID NO: 130, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, and SEQ ID NO: 149.

13. The pan-coronavirus immunogenic composition of claim 1, wherein said coronavirus monoclonal antibody comprises a light chain, and wherein said light chain comprises one or more selected from the group consisting of:
a) a light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152;
b) a polypeptide comprising an amino acid sequence at least 90% identical to one or more light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152;
c) a polypeptide comprising an amino acid sequence at least 70% of the length of one or more light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152; and
d) a polypeptide comprising an amino acid sequence at least 90% identical to and at least 70% the length of one or more light chain amino acid sequence selected from the group consisting of: SEQ ID NO: 119, SEQ ID NO: 120, SEQ ID NO: 123, SEQ ID NO: 124, SEQ ID NO: 127, SEQ ID NO: 128, SEQ ID NO: 131, SEQ ID NO: 132, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 150, SEQ ID NO: 151, and SEQ ID NO: 152.

14. The pan-coronavirus immunogenic composition comprising one or more coronavirus monoclonal antibody of claim 1, wherein said composition comprises a bispecific antibody comprising a heavy chain IgG Fc “knob”, a heavy chain IgG Fc “hole”, and at least two light chains.

15. The pan-coronavirus immunogenic composition comprising one or more coronavirus monoclonal antibody of claim 14, wherein:
a) said heavy chain IgG Fc “knob” comprises an amino acid sequence selected from the group consisting of: SEQ ID NO: 134, SEQ ID NO: 135, SEQ ID NO: 136, SEQ ID NO: 137, SEQ ID NO: 138, and SEQ ID NO: 139;
b) said heavy chain IgG Fc “hole” comprises the amino acid sequence of SEQ ID NO: 133; and
c) said at least two light chains comprise at least two amino acid sequences selected from the group consisting of: SEQ ID NO: 140, SEQ ID NO: 141, SEQ ID NO: 142, SEQ ID NO: 143, SEQ ID NO: 144, SEQ ID NO: 145, and SEQ ID NO: 146.

16. A method of administering to a subject in need thereof one or more pan-coronavirus immunogenic composition, comprising administering the subject one or more selected from the group consisting of:
a) one or more coronavirus antigenic polypeptide;
b) one or more nucleic acid molecule encoding one or more coronavirus antigenic polypeptide;
c) one or more coronavirus monoclonal antibody; and
d) one or more nucleic acid molecule encoding one or more coronavirus monoclonal antibody.

17. A method of treating or preventing infection by more than one human coronavirus in a subject, comprising administering to the subject one or more selected from the group consisting of:
a) one or more coronavirus antigenic polypeptide;
b) one or more nucleic acid molecule encoding one or more coronavirus antigenic polypeptide;
c) one or more coronavirus monoclonal antibody; and
d) one or more nucleic acid molecule encoding one or more coronavirus monoclonal antibody.

18. The method of claim 17, wherein said subject is a human.

19. The method of claim 18, wherein the subject is infected with one or more human coronavirus or at risk of becoming infected with one or more human coronavirus.

20. The method of claim 19, wherein the human coronavirus is one or more selected from the group consisting of: 229E, NL63, OC43, HKU1, MERS-CoV, SARS-CoV, and SARS-CoV-2.
